(19) United States
(12) Patent Application Publication
(10) Pub. No.: US 2017/0296457 A1
(43) Pub. Date: Oct. 19, 2017

(54) PRODUCTS AND METHODS FOR TREATING PERIORBITAL DYSCHROMIA

(71) Applicant: The Procter & Gamble Company, Cincinnati, OH (US)

(72) Inventor: Karen Marie OSORIO, CIncinnati, OH (US)

(21) Appl. No.: 15/098,804

(22) Filed: Apr. 14, 2016

(51) Int. Cl. A61K 8/97; A61K 8/60; A61K 8/44; A61K 8/64; A61K 8/06; A61K 8/49; A61Q 19/02; A61K 8/368

(52) U.S. Cl. CPC A61K 8/97; A61Q 19/02; A61K 8/602; A61K 8/368; A61K 8/64; A61K 8/062; A61K 8/4926; A61K 8/442; A61K 2800/592; A61K 2800/87

(57) ABSTRACT

The present application is directed, generally, to an array of products and methods for treating periorbital dyschromia. More specifically, the present application discloses an array of products and methods for treating different types of periorbital dyschromia. The products and methods utilize extracts of Cucurbita pepo.

### FIELD OF THE INVENTION

The present application is directed, generally, to an array of products and methods for treating periorbital dyschromia. More specifically, the present application discloses an array of products and methods for treating different types of periorbital dyschromia. The products and methods utilize extracts of Cucurbita pepo.

### BACKGROUND OF THE INVENTION

A person's eyes are a prominent and noticeable facial feature. Thus, any desirable or undesirable aesthetic features associated with the eyes may influence an individual's perception of herself or himself or the impression that the individual makes on others. Undesirable aesthetic features may include lines, wrinkles and discoloration of the skin. For example, some people may find periorbital dyschromia, sometimes referred to as dark circles or under-eye dark circles, to be aesthetically undesirable and/or they may associate the appearance of periorbital dyschromia with fatigue and/or age. It should come as no surprise then that throughout history a variety of ways to accentuate and/or beautify the eyes have been devised. A common approach to improve the appearance of periorbital dyschromia is to use a cosmetic composition such as a concealer to hide the discoloration. Using make up to hide a perceived flaw may provide a temporary cosmetic benefit, but most conventional make up products require daily application and, in some instances, may even require reapplication throughout the day. Thus, a more permanent solution is desired to reduce and/or eliminate some of the undesirable aesthetic features commonly found around the eye.

U.S. patent application Ser. No. 14/472,716, published May 7, 2015, by Osorio et al., the disclosure of which is incorporated herein by reference, provides a thorough background discussion of periorbital dyschromia, including a summary of specific types of the disorder and different theories explaining causes of the disorder. The application also discloses methods of identifying and evaluating potential cosmetic agents, and discusses gene expression signatures, data architectures and connectivity mapping relating to different types of periorbital dyschromia. The discussion in U.S. patent application Ser. No. 14/472,716 of periorbital dyschromia is useful in understanding the arrays and methods described herein.

### BRIEF DESCRIPTION OF THE DRAWINGS

FIG. 1 is an illustration of various portions of a human face.

FIGS. 2A and 2B illustrate examples of the portion of the periorbital region affected by Type I periorbital dyschromia.

FIGS. 3A and 3B illustrate examples of portion of the periorbital region affected by Type II periorbital dyschromia.

FIGS. 4A and 4B illustrate examples of portion of the periorbital region affected by Type III periorbital dyschromia.

FIGS. 5A, 6A and 7A are images of a test subject captured with a digital camera.

FIGS. 5B, 6B and 7B are Brown channel images of a test subject.

FIGS. 5C, 6C and 7C are Red channel images of a test subject.

FIGS. 5D, 6D and 7D illustrate the location of the periorbital dyschromia seen in FIGS. 5B, 6B and 7B, respectively.

FIGS. 5E, 6E and 7E illustrate the location of the periorbital dyschromia seen in FIGS. 5C, 6C and 7C, respectively.

FIG. 8 is a 20× micrograph of a skin tissue sample from a subject exhibiting No Dyschromia. “Epi” signifies the epidermal layer and “Der” the dermal layer of the tissue sample.

FIG. 9 is a 20× micrograph of a skin tissue sample from a subject exhibiting Type I periorbital dyschromia.

FIG. 10 is a 20× micrograph of a skin tissue sample from a subject exhibiting Type II periorbital dyschromia.

FIG. 11 is a 20× micrograph of a skin tissue sample from a subject exhibiting Type III periorbital dyschromia.

FIG. 12 illustrates the Fontana-Masson positive bodies observed per field of view for each type of periorbital dyschromia and a No Dyschromia condition.

FIG. 13 illustrates the normalized PTCA amount measured for each type of periorbital dyschromia and a No Dyschromia condition.

FIG. 14 illustrates an example of a masked region corresponding to Zone 1.

FIG. 15 illustrates an example of a masked region corresponding to Zone 2.

FIG. 16 illustrates an example of a masked region corresponding to Zone 4.

FIG. 17 illustrates an example of a masked region corresponding to the cheek.

FIG. 18 illustrates an example of a masked region corresponding to the cheek.

FIG. 19 shows a comparison of the R values for different types of periorbital dyschromia in Zone 1.

FIG. 20 is a comparison of the B values for different types of periorbital dyschromia in Zone 1.

FIG. 21 is a comparison of the G values for different types of periorbital dyschromia in Zone 1.

FIG. 22 is a comparison of the B/G ratios for different types of periorbital dyschromia in Zone 1.

FIG. 23 is a comparison of the R values for different types of periorbital dyschromia in Zone 2.

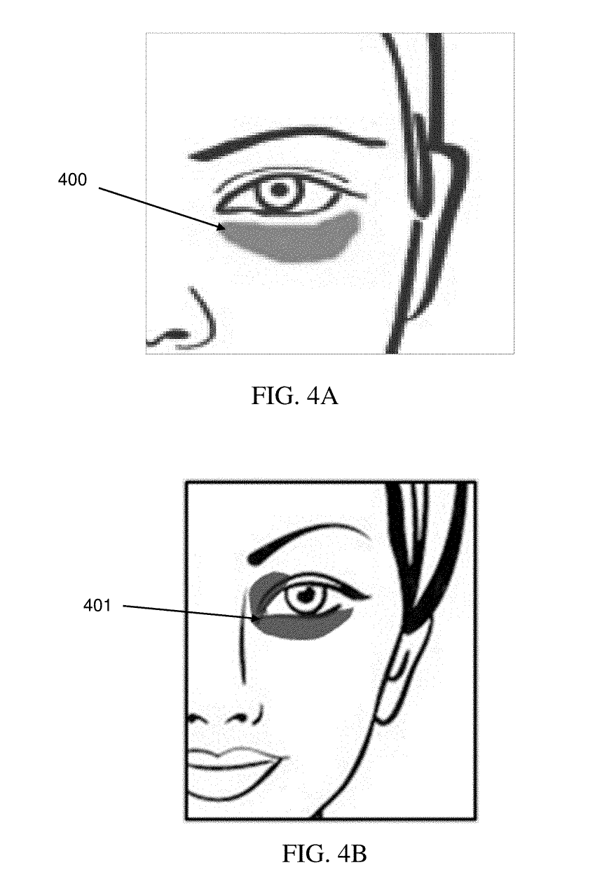

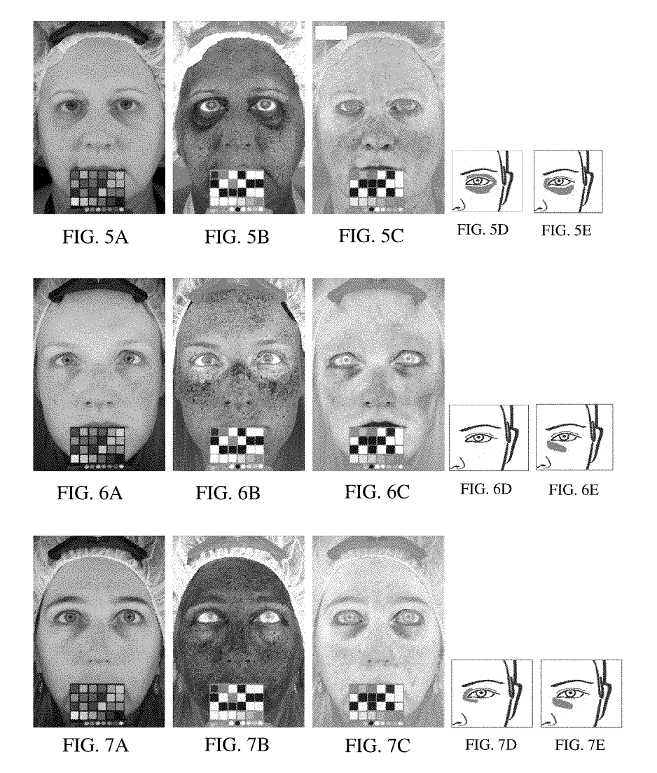

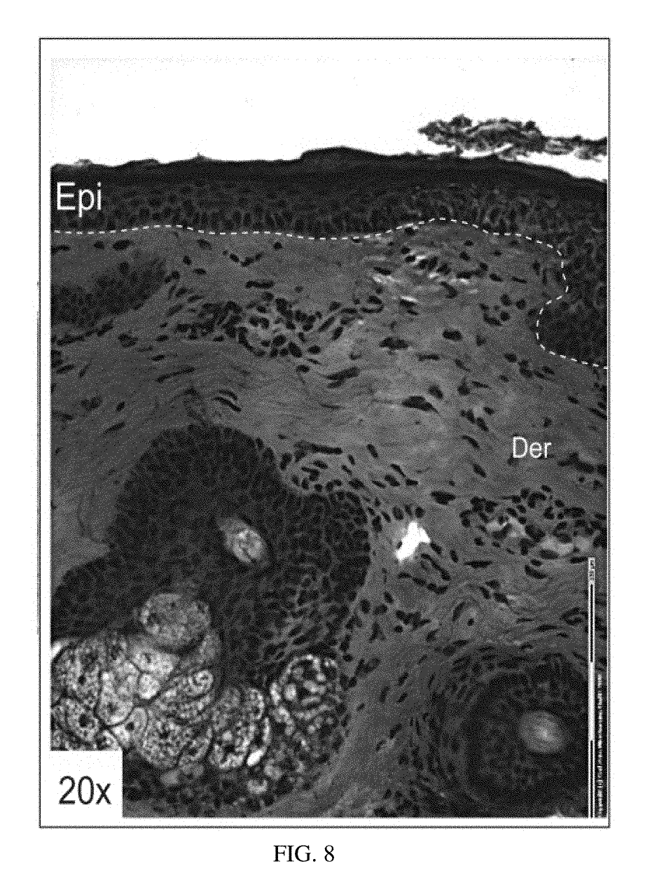

FIG. 4 is a comparison of the G values for different types of periorbital dyschromia in Zone 2

FIG. 25 is a comparison of the B values for different types of periorbital dyschromia in Zone 2.

FIG. 26 is a comparison of the B/G ratios for different types of periorbital dyschromia in Zone 2.

FIG. 27 is a comparison of the R values for different types of periorbital dyschromia in Zone 4 (i.e., Zones 1 and 2, in combination).

FIG. 28 is a comparison of the G values for different types of periorbital dyschromia in Zone 4 (i.e., Zones 1 and 2, in combination).

FIG. 29 is a comparison of the B values for different types of periorbital dyschromia in Zone 4 (i.e., Zones 1 and 2, in combination).

FIG. 30 is a comparison of the B/G ratios for different types of periorbital dyschromia in Zone 4 (i.e., Zones 1 and 2, in combination).

### DETAILED DESCRIPTION OF THE INVENTION

All percentages are by weight of the personal-care composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements. All measurements are understood to be made at about 25° C. and at ambient conditions, where “ambient conditions” means conditions under about one atmosphere of pressure and at about 50% relative humidity.

### Definitions

“Applicator” means a device used to apply a personal care composition to a target surface.

“Chronic active” means an active suitable for use in a topical cosmetic composition that continues to provide the desired benefit after use of the active is discontinued. Chronic actives provide a relatively long lasting cosmetic benefit as compared to the acute actives commonly found in conventional makeup products that are intended to cover or hide perceived cosmetic flaws (e.g., the pigments, dyes, lakes and other colorants commonly found in foundations and concealers). In some instances, chronic actives work via recurrent use of the active over an extended period of time (e.g., use of the active for more than 1 week). In contrast, acute actives have no lasting effect on the skin, and once the acute active is removed, the skin is the same in appearance as before the acute active was applied. Compositions containing chronic actives may be applied on the order of about once per day over such extended periods. In some instances, the application rates may vary from about once per week to about three times per day or at some rate in between. The chronic active may provide the desired benefit almost immediately, or after some minimum amount of recurring use of die composition (e.g., after 1, 2, 3, 4, 5, 6, 7, 8, 9, 19, 11, or even 12 weeks). The benefit provided by the chronic active may last for more than 1 day (e.g., more than 2, 3, 4, 5, or 6 days), more than I week (e.g., more than 2, 3, or 4 weeks) or even more than a month after use of the composition containing the chronic active is discontinued.

“Cosmetic” means providing a desired visual effect on an area of the human body. The visual cosmetic effect may be temporary, semnpermanent, or permanent. Some non-limiting examples of “cosmetic products” include products that leave color on the face, such as foundation, mascara, concealers, eye liners, brow colors, eye shadows, blushers, lip sticks, lip balms, face powders, solid emulsion compact, and the like.

“Cosmetic agent” means any substance, as well any component thereof, intended to be rubbed, poured, sprinkled, sprayed, introduced into, or otherwise applied to a mammalian body or any part thereof to provide a cosmetic benefit. Cosmetic agents may include substances that are Generally Recognized as Safe (GRAS) by the US Food and Drug Administration, food additives, and materials used in non-cosmetic consumer products including over-the-counter medications. In some embodiments, cosmetic agents may be incorporated in a cosmetic composition comprising a dermatologically acceptable carrier suitable for topical application to skin. A cosmetic agent includes, but is not limited to, (i) chemicals, compounds, small or large molecules, extracts, formulations, or combinations thereof that are known to induce or cause at least one effect (positive or negative) on skin tissue; (ii) chemicals, compounds, small molecules, extracts, formulations, or combinations thereof that are known to induce or cause at least one effect (positive or negative) on skin tissue and are discovered, using the provided methods and systems, to induce or cause at least one previously unknown effect (positive or negative) on the skin tissue; (iii) chemicals, compounds, small molecules, extracts, formulations, or combinations thereof that are not known have an effect on skin tissue and are discovered, using the provided methods and systems, to induce or cause an effect on skin tissue; and (iv) a compound or combination of compounds that, when applied to skin, provide an acute and/or chronic benefit to skin or a type of cell commonly found therein. Cosmetic agents may regulate and/or improve skin or its associated cells (e.g., improve skin elasticity; improve skin hydration; improve skin condition; and improve cell metabolism).

Some examples of cosmetic agents or cosmetically actionable materials can be found in: the PubChem database associated with the National Institutes of Health, USA (http://pubchem.ncbi.nlm nih gov); the Ingredient Database of the Personal Care Products Council (http://online. personalcarecouncil.org/jsp/Home.jsp); and the 2010 International Cosmetic Ingredient Dictionary and Handbook, 13^{th} Edition, published by The Personal Care Products Council; the EU Cosmetic Ingredients and Substances list; the Japan Cosmetic Ingredients List; the Personal Care Products Council, the SkinDeep database (URL: http://www.cosmeticsdatabase.com); the FDA Approved Excipients List; the FDA OTC List; the Japan Quasi Drug List; the US FDA Everything Added to Food database; EU Food Additive list; Japan Existing Food Additives, Flavor GRAS list; US FDA Select Committee on GRAS Substances; US Household Products Database; the Global New Products Database (GNPD) Personal Care, Health Care, Food/Drink/Pet and Household database (URL: http://www.gnpd.com); and from suppliers of cosmetic ingredients and botanicals.

Other non-limiting examples of cosmetic agents include botanicals (which may be derived from one or more of a root, stem bark, leaf, seed or fruit of a plant). Some botanicals may be extracted from a plant biomass (e.g., root, stem, bark, leaf, etc.) using one more solvents. Botanicals may comprise a complex mixture of compounds and lack a distinct active ingredient. Another category of cosmetic agents are vitamin compounds and derivatives and combinations thereof, such as a vitamin B3 compound, a vitamin B5 compound, a vitamin B6 compound, a vitamin B9 compound, a vitamin A compound, a vitamin C compound, a vitamin E compound, and derivatives and combinations thereof (e.g., retinol, retinyl esters, niacinamide, folic acid, panthenol, ascorbic acid, tocopherol, and tocopherol acetate). Other non-limiting examples of cosmetic agents include sugar amines, phytosterols, hexamidine, hydroxy acids, ceramides, amino acids, and polyols.

“Cosmetic composition” means a composition that is intended to be applied to a bodily surface to provide an aesthetic benefit. Cosmetic compositions typically include a cosmetic agent, but need not necessarily do so. Cosmetic composition include make up and skin care compositions.

“Disposed” refers to an element being located in a particular place or position relative to another element.

“Effective amount” means an amount of a compound or composition sufficient to significantly induce a positive or desired benefit, (e.g., a positive skin or feel benefit, reverse the expression of a gene, group of genes and/or gene signature), including independently or in combinations the benefits disclosed herein, but low enough to avoid serious side effects, i.e., to provide a reasonable benefit to risk ratio, within the scope of sound judgment of the skilled artisan.

“Joined” means configurations whereby an element is directly secured to another element by affixing the element directly to the other element, and configurations whereby an element is indirectly secured to another element by affixing the element to intermediate member(s) that in turn are affixed to the other element.

“Keratinous tissue,” means keratin-containing tissue layers disposed as the outermost protective covering of mammals which includes, but is not limited to, skin, hair, and nails.

“Keratinous tissue,” means a direction that is generally perpendicular to an imaginary centerline that bisects the human body into right and left mirror images. Directions that are within forty-five degrees of being perpendicular to the imaginary centerline are considered lateral.

“Longitudinal” means a direction that is generally parallel to an imaginary centerline that bisects the human body into right and left mirror images. Directions that are within forty-five degrees of being parallel to the imaginary centerline are considered longitudinal.

“Periorbital” means around the orbit of the eye. The periorbital region of a person is the area of the face generally disposed around the eye socket and typically lies between the bottom of the brow and the top of the cheek in the longitudinal direction and between. the bridge of the nose and the temple in the lateral direction.

“Periorbital dyschromia” is a condition that occurs when the tone of the skin in the periorbital region of person is noticeably different from tone of the skin in a nearby portion of the face such as the cheek, nose, forehead, temple and/or another portion of the periorbital region. Perioribital dyschromia is generally bilateral (i.e., it occurs in the periorbital region of both sides of the face). Periorbital dyschromia may manifest as the appearance of a difference in skin tone in the periorbital region relative to other regions of the face and/or body (e.g., cheek, nose, forehead, temple, chin, arm or leg). Periorbital dyschromia may appear as a result of hyperpigmented or hypopigmented skin in the periorbital region.

“Personal care composition” means a cosmetic composition or a skin care composition. Is it to be appreciated that a personal care composition may provide both a cosmetic benefit and a skin health benefit.

“Skin” means the outermost protective covering of mammals that is composed of cells such as keratinocytes, fibroblasts and melanocytes. Skin includes an outer epidermal layer and an underlying dermal layer. Skin may also include hair follicles, sebaceous gland and nails as well as other types of cells commonly associated with skin, such as, for example, myocytes, Merkel cells, Langerhans cells, macrophages, stem cells, sebocytes, nerve cells and adipocytes.

“Skin care” means regulating and/or improving skin condition. Some nonlimiting examples of skin care benefits include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin texture or smoothness, improving skin exfoliation or desquamation, plumping the skin, improving skin barrier properties, improve skin tone, reducing the appearance of redness or skin blotches, and/or improving the brightness, radiance, or translucency of skin. Some nonlimiting examples of “skin care products” include skin creams, moisturizers, lotions, and body washes.

“Skin-care composition” means a composition that regulates and/or improves skin condition.

“Skin tone” refers to the perceived color or pigmentation of skin, especially with regard to the evenness of the coloration or pigmentation. “Skin tone” may also include other characteristics of skin that contribute to a consumer perception of overall tone. For example, pore size and distribution, and skin texture may also be considered attributes of overall skin tone.

“Software” and “software application” mean one or more computer readable and/or executable instructions that cause a computing device or other electronic device to perform functions, actions, and/or behave in a desired manner. The instructions may be embodied in one or more various forms like routines, algorithms, modules, libraries, methods, and/or programs. Software may be implemented in a variety of executable and/or loadable forms and can be located in one computer component and/or distributed between two or more communicating, co-operating, and/or parallel processing computer components and thus can be loaded and/or executed in serial, parallel, and other manners. Software can be stored on one or more computer readable medium and may implement, in whole or part, the methods and functionalities of the present invention.

“Topical application” means to apply or spread the compositions of the present invention onto the surface of the keratinous tissue.

Previously, the underlying causes of periorbital dyschromia were not particularly well elucidated. However, as discussed, it has unexpectedly been found that there are common themes associated with periorbital dyschromia that can lend themselves to differentiation based on a variety of relatively straightforward evaluation techniques. Previous attempts to classify periorbital dyschromia did not appreciate that periorbital dyschromia can be grouped into distinct categories based on the visual evaluation imaging, biomarkers, histology, and/or genetic analysis techniques disclosed herein. Based on these newly discovered distinctions, it is believed that the array of products described herein may provide tailored solutions for treating different types of periorbital dyschromia.

Furthermore, the discovery that there are different types of periorbital dyschromia with different underlying biological causes and appearances has led to a need to identify chronic actives and/or combinations of actives that can provide tailored solutions for treating each of the different types of periorbital dyschromia. It has surprisingly been found that an extract of Cucurbita pepo, sometimes referred to as pumpkin seed, can improve the appearance of periorbital dyschromia, and in particular Type II and/or Type III periorbital dyschromia. While pumpkin seed extract (INCI name: Sea Water (and) Water (and) Cucurbita pepo (pumpkin) seed extract; CAS No.89998-03-5) is known for, among other things, use in treating dry, inflamed skin, wounds, rashes, and wrinkles , it was not previously known that pumpkin seed extract can be used to improve the appearance of periorbital dyschromia. In addition, our studies suggest that pumpkin seed extract may not adversely affect the appearance of Type I periorbital dyschromia, which is especially desirable in the event a consumer's periorbital dyschromia type is misidentified and pumpkin seed extract is used to treat it.

### Classifying Periorbital Dyschromia

Periorbital dyschromia may be classified into different types using a variety of evaluation techniques such as, for example, visual evaluation, imaging analysis, histological analysis, biomarker analysis, gene expression signature analysis and/or gene expression theme analysis. Suitable examples of systems and methods of classifying periorbital dyschromia and descriptions of Type I, Type II, Type III and “No Dyschromia” are described in U.S. Provisional App. No. 61/798,208, filed on Mar. 15, 2013 by Osorio, et al., and titled “Methods of Classifying Periorbital Dyschromia and Systems Therefor.” The classification of periorbital dyschromia described in some embodiments below (e.g., the imaging values and ratios) is in relation to Fitzpatrick I, II and III skin types, according to the commonly known Fitzpatrick Scale. However, it is believed, without being limited by theory, that the classification methods and systems herein can be reapplied to people with Fitzpatrick IV, V and VI skin types in the substantially the same manner For example, by comparing the imaging values of one or more zones in the periorbital region to a reference or control value (e.g., the imaging values associated with the cheek), a differential color value can be determined, which may be used to identify the type of periorbital dyschromia present.

However, the array of products described herein is not limited to use with Fitzpatrick I, II and/or III skin types, but may be used in conjunction with any skin type (e.g., Fitzpatrick IV, V and VI), based on a suitable definition of periorbital dyschromia associated with the particular skin type(s).

In some instances, Type I, Type II, Type III and No Dyschromia may be distinguished from one another visually or with an imaging system. In some instances, it may be desirable to use an expert grader (i.e., someone trained to visually classify periorbital dyschromia) to visually classify the type of periorbital dyschromia either in-person or from a captured image. Additionally or alternatively, visual classification may be done by a non-expert (e.g., a consumer who self-diagnoses) based on, for example, a set of instructions or a visual cue. A suitable method of visually classifying periorbital dyschromia is described in more detail in the Methods section below. In some embodiments, a No Dyschromia condition may be visually characterized by the lack of an uneven or discontinuous skin tone in the periorbital region. In some embodiments, Type I periorbital dyschromia may be visually characterized by continuous discoloration of both the upper and lower eyelid skin. The discolored periorbital skin associated with Type I periorbital dyschromia typically includes substantially uniform brown, yellow and/or orange tones in the skin of the periorbital region, which may resemble the color of tanned skin or an age spot. Type I periorbital dyschromia may also be generally defined, in part, by its location in the upper and lower portions of the periorbital region of the face (e.g., proximate the lower eyelid and the upper eyelid). Type II periorbital dyschromia may be characterized by continuous discoloration of the lower eyelid skin. The discolored periorbital skin associated with Type II periorbital dyschromia typically includes substantially uniform purple, pink and/or bluish tones, which may resemble the color of bruised skin. Type II is generally defined, in part, by its presence in the inner, lower portion of the periorbital region and its absence in the upper portion (i.e., upper eyelid or Zone 3) and outer, lower portion (i.e., Zone 2). In other words, Type II dyschromia is typically found proximate the lower eyelid and the bridge of the nose. Type III periorbital dyschromia may be characterized by the presence of skin tones that resemble sunburned skin. Type III is generally defined, in part, by its presence in the under-eye and above-the-eye portions of the periorbital region.

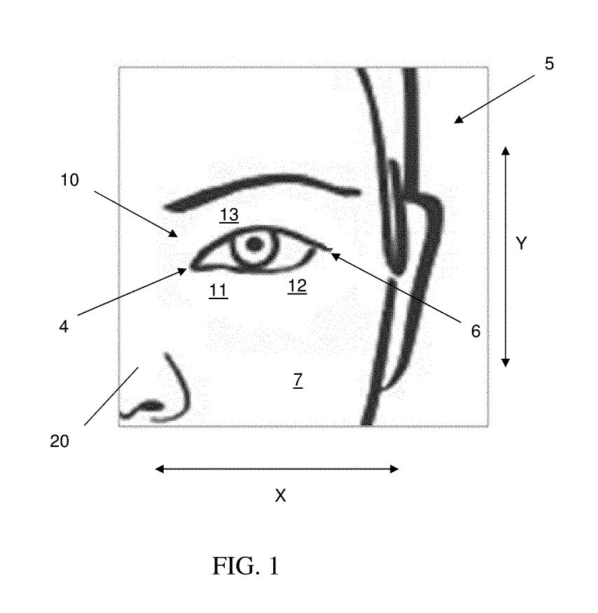

FIG. 1 illustrates the periorbital region 10 of a human face 5 divided into three zones. Zone 1 11 is disposed generally under the eye in the longitudinal direction Y and extends in the lateral direction X from the inside corner 4 of the eye (i.e., the corner of the eye disposed closest to the nose 20) to an area under the eye that is less than the complete distance to the outside corner 6 of the eye (i.e., the corner of the eye disposed furthest from the nose). In some embodiments, as illustrated in FIG. 1, Zone 1 11 may extend approximately to the middle of the eye in the lateral direction X. But it is to be appreciated that, in some individuals, Zone 1 may extend more than halfway or less than halfway across the under-eye region, but typically not more than 90% of the corner-to-corner distance under the eye. Zone 2 12 extends from the distal edge of Zone 1 11 (i.e., the edge furthest from the nose 20) to the outside corner 6 of the eye. Zone 1 11 and Zone 2 12, which are collectively referred to as Zone 4 (not labeled on figure), extend generally in the longitudinal direction Y from the bottom of the eye, which includes the lower eyelid, to the top of the cheekbone. Zone 3 is disposed above the eye and extends laterally from the inside corner 4 of the eye to the outside corner 6 of the eye (i.e., Zone 3 13 extends the entire corner-to-corner distance above the eye). Zone 3 13 also extends generally in the longitudinal direction Y from the top of the eye, including the upper eyelid, to the eyebrow. In some embodiments, it may be desirable to use the cheek 7 of the face 5 as a reference or control when evaluating the location and/or tones of the periorbital dyschromia.

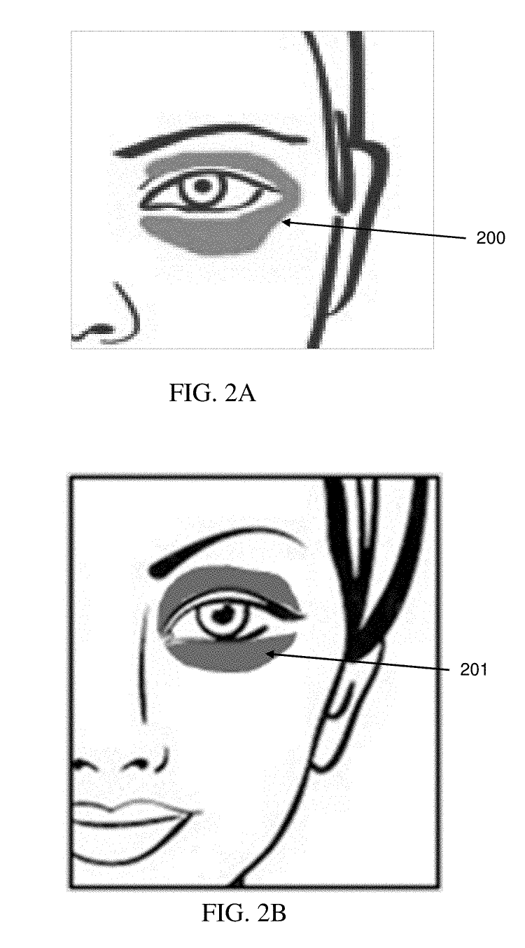

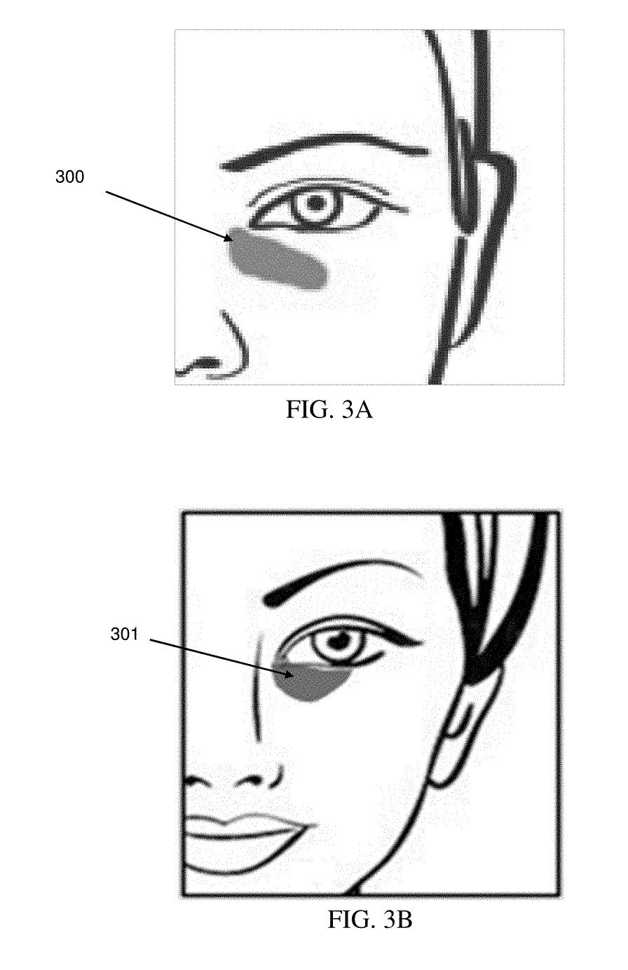

FIGS. 2A and 2B illustrate examples of Type I periorbital dyschromia, which is represented by the shaded portions 200 and 201, respectively, of the periorbital region. FIGS. 3A and 3B illustrate examples of Type II periorbital dyschromia (i.e., the shaded portions 300 and 301, respectively, of the periorbital region). FIGS. 4A and 4B illustrate examples of Type III periorbital dyschromia (i.e., the shaded portions 400 and 401, respectively, of the periorbital region).

Type I, Type II and Type III periorbital dyschromia may be distinguished from one another using known imaging technique such as RGB and/or RBX® color imaging and/or gray scale imaging. An image of the region of interest (“ROI”) (e.g., periorbital region of a person) is captured by an image capture device, and at least a portion of the image is analyzed (e.g., by a computer) and assigned an imaging value based on the analysis. This determined imaging value may then be compared to a predetermined imaging value, which can be a single value or a range of values (e.g., one or more of the RGB values disclosed herein). The predetermined imaging value defines a particular type of periorbital dyschromia, and the comparison enables a user to identify which type of periorbital dyschromia, if any, is exhibited by the person. It may be desirable to color correct the images to compensate for instrument variability and/or normalize the imaging values to compensate for variability based on skin tone.

Type I periorbital dyschromia may be characterized by generally having lower RGB values relative to Types II and III as well as a different ratio of B value to G value (“B/G”). Type II periorbital dyschromia may be characterized by generally having higher RGB values compared to Types I and III. Type III periorbital dyschromia may include characteristics of both Type I and Type II. Tables 1, 2 and 3 below show ranges of color corrected RGB values, B/G ratios, L*a*b* values, chroma values and hue values that may be used to characterize Type I, Type II and Type III periorbital dyschromia, respectively, exhibited by a person having a Fitzpatrick skin type of I, II or III. The values provided in Tables A, B and C correspond to a particular portion of the periorbital region referred to as a “mask.” Masks, and in particular Mask A, Mask B and Mask C are described in more detail below in the Imaging Method, which provides a suitable method of determining imaging values.

TABLE A
Type I

| Imaging Value | Mask A | Mask B | Mask C |
|---|---|---|---|
| R | 118-166 | 135-187 | 128-178 |
| G | 83-119 | 94-136 | 89-129 |
| B | 76-108 | 81-112 | 79-112 |
| B/G | 0.75-0.92 | 0.73-0.86 | 0.74-0.89 |
| L | 48-54 | 43-61 | 41-59 |
| A | 13-18 | 9-17 | 12-18 |
| b | 9-23 | 14-25 | 12-23 |
| Hue (h) | 35-53 | 42-60 | 39-56 |
| Chroma (C) | 16-28 | 17-28 | 17-28 |

TABLE B
Type II

| Imaging Value | Mask A | Mask B | Mask C |
|---|---|---|---|
| R | 148-176 | 165-201 | 159-186 |
| G | 102-135 | 128-160 | 115-148 |
| B | 96-128 | 108-145 | 103-136 |
| B/G | 0.86-0.98 | 0.82-0.93 | 0.84-0.94 |
| L | 48-60 | 57-69 | 52-64 |
| a | 11-20 | 9-19 | 10-19 |
| b | 7-16 | 10-20 | 9-17 |
| Hue (h) | 24-54 | 33-61 | 32-57 |
| Chroma (C) | 15-23 | 15-22 | 15-22 |

TABLE C
Type III

| Imaging Value | Mask A | Mask B | Mask C |
|---|---|---|---|
| R | 142-172 | 160-188 | 153-177 |
| G | 97-129 | 110-148 | 105-137 |
| B | 88-114 | 92-129 | 90-119 |
| B/G | 0.81-0.95 | 0.79-0.89 | 0.80-0.92 |
| L | 46-57 | 51-64 | 49-60 |
| a | 11-20 | 10-17 | 11-18 |
| b | 9-19 | 13-21 | 11-19 |
| Hue (h) | 29-54 | 42-62 | 36-57 |
| Chroma (C) | 17-24 | 18-24 | 18-24 |

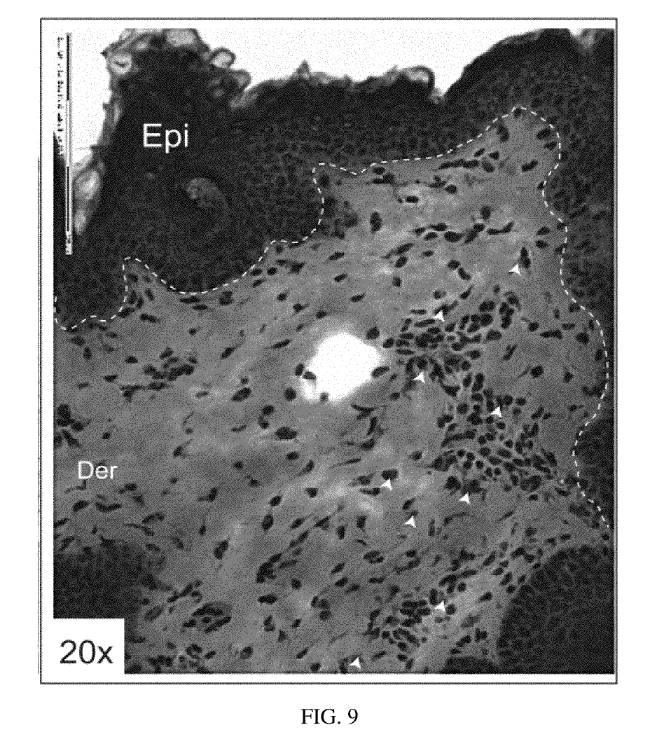

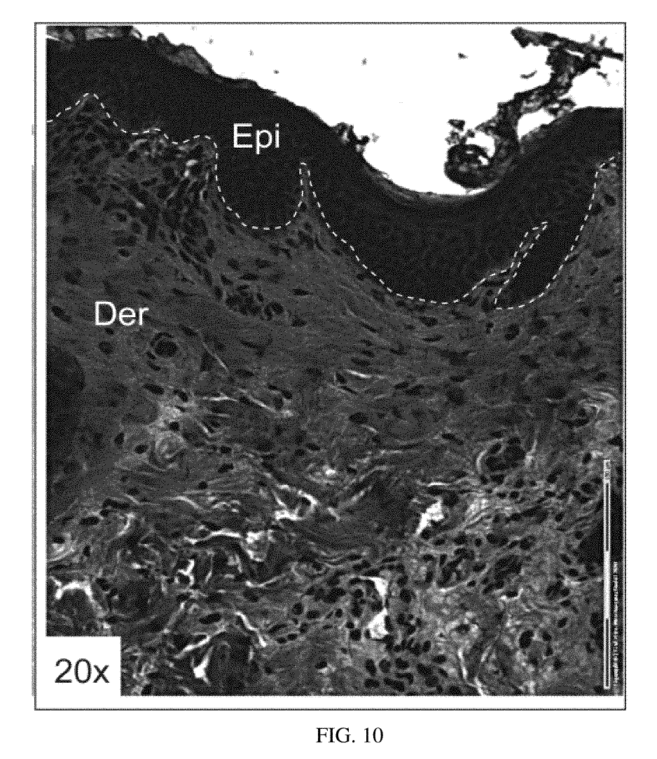

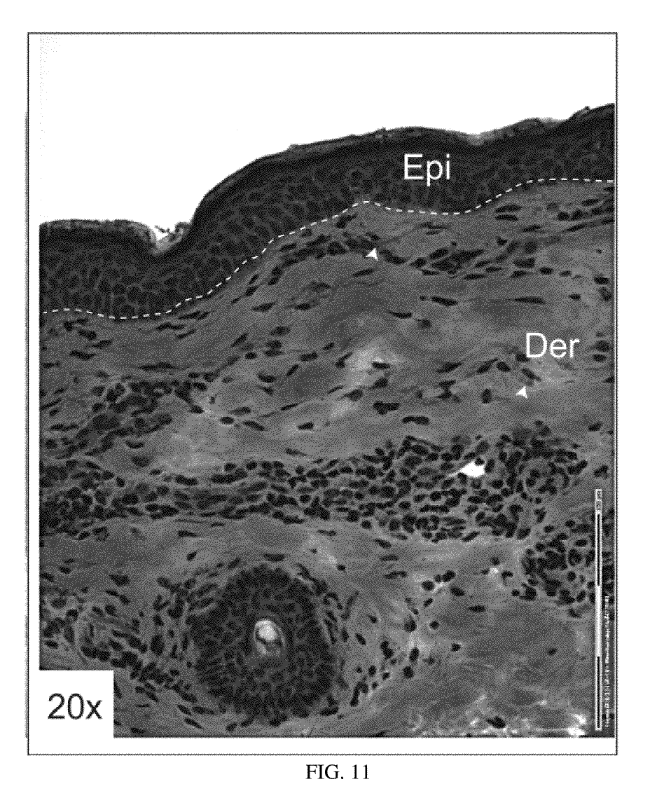

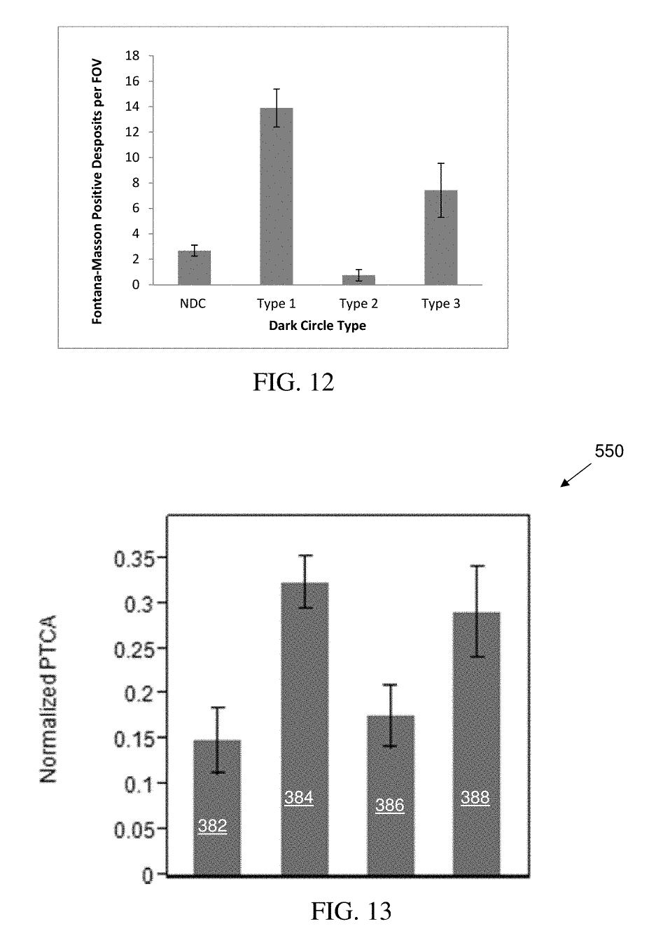

FIGS. 9 through 11 illustrate visually the data summarized graphically in FIG. 12.

FIG. 12 illustrates the Fontana-Masson positive bodies observed per field of view for each type of periorbital dyschromia and a No Dyschromia condition (NDC). FIG. 12 reflects significantly greater deposits with Type I, and significantly fewer deposits for Type II and III compared to NDC.

FIG. 13 illustrates the comparison between pyrrole-2,3,5-tricarboxylic acid (“PTCA”) levels for the different types of periorbital dyschromia. The data reflected in FIG. 13 are discussed in detail below.

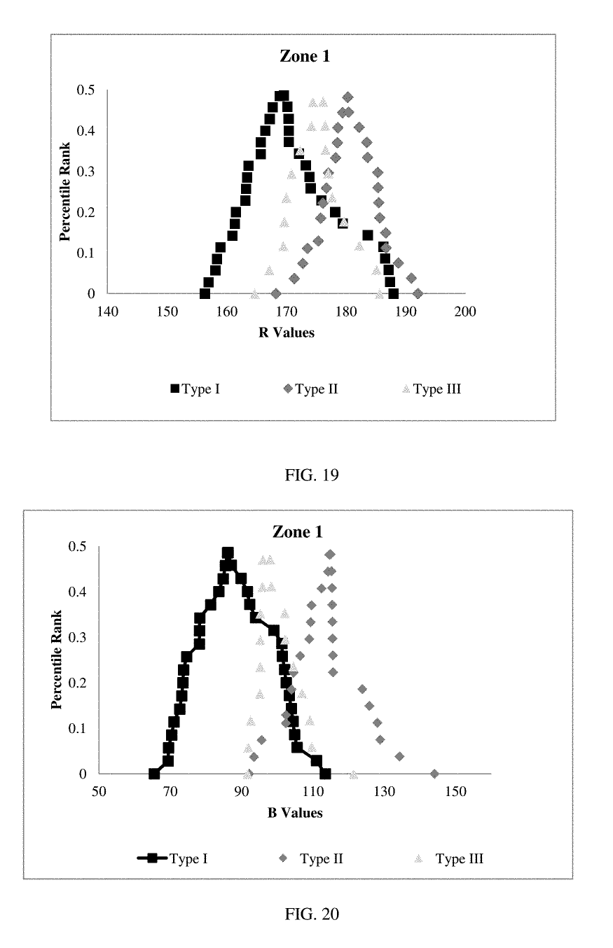

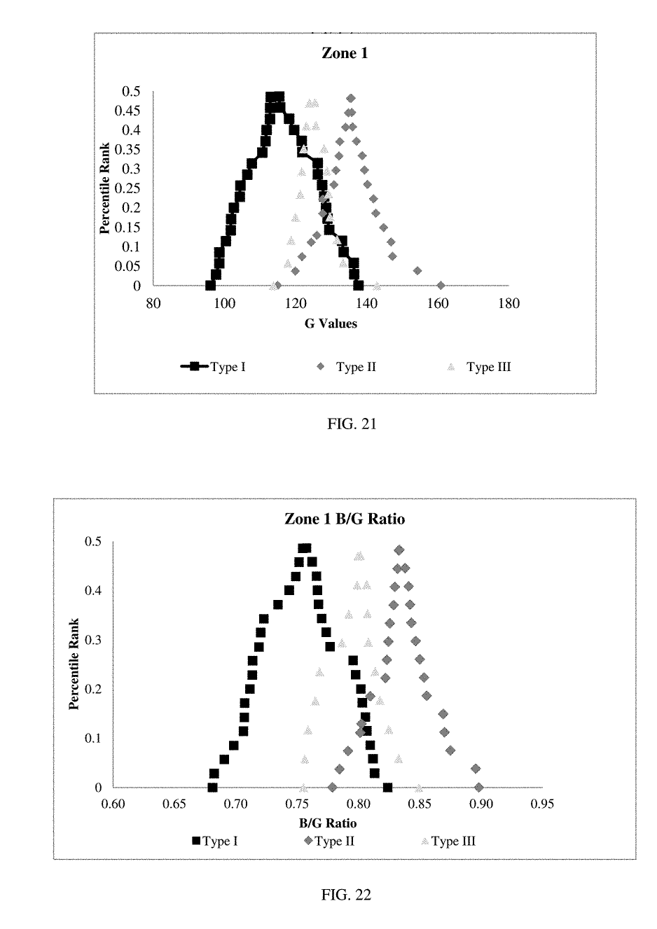

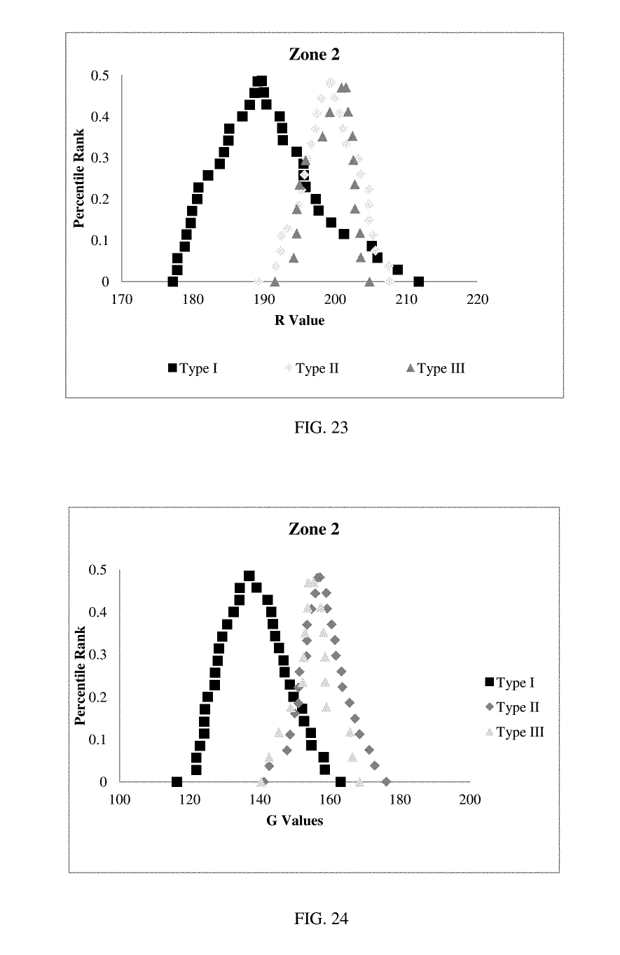

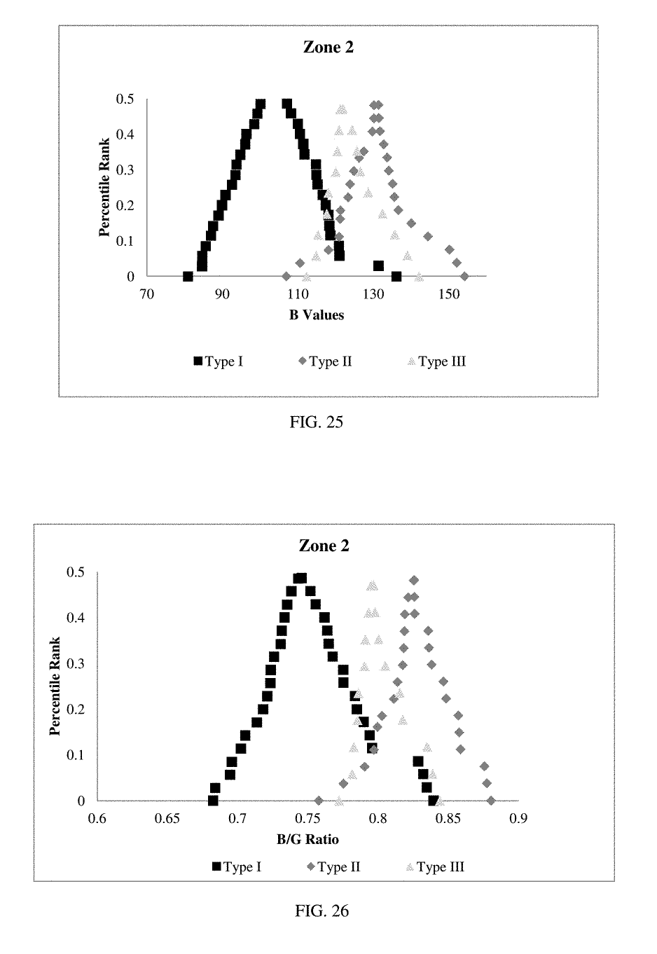

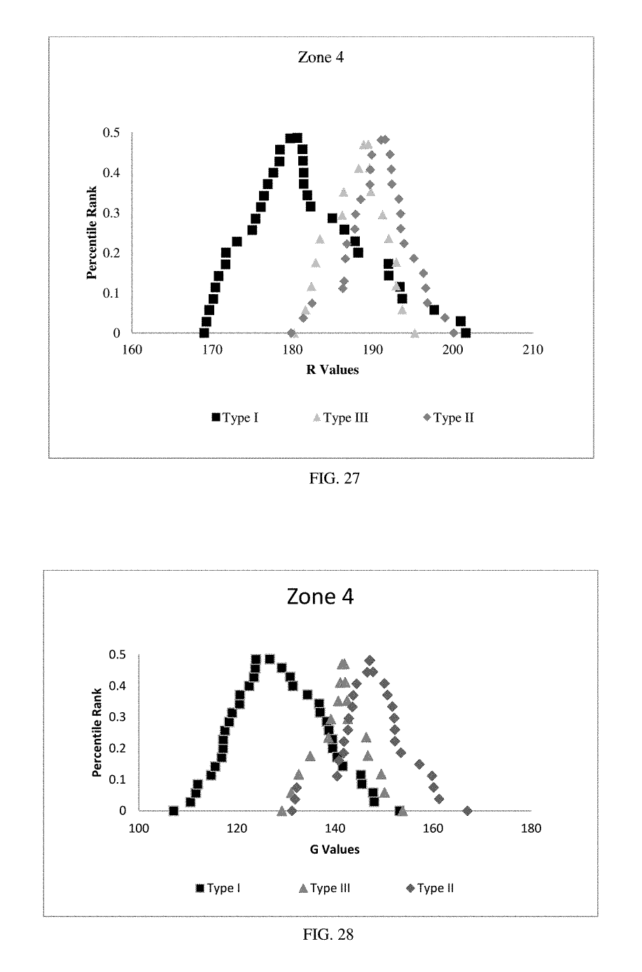

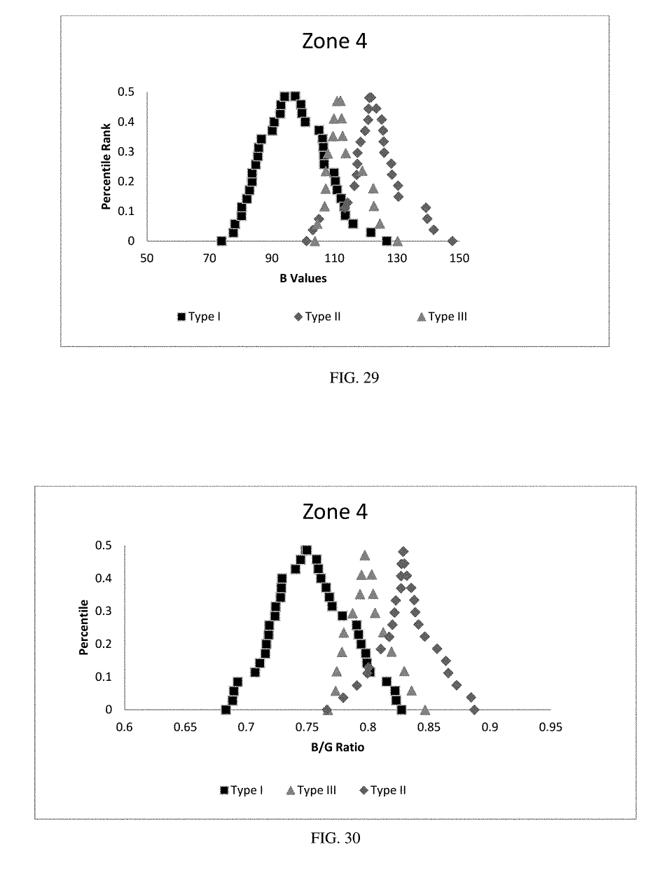

FIGS. 19 through 30 are side-by-side comparisons of graphical plots representing imaging values corresponding to Type I, Type II and Type III test subjects. FIG. 19 shows a side-by-side comparison of Zone 1 R values. FIG. 20 shows a side-by-side comparison of the Zone 1 B values. FIG. 21 shows a side-by-side comparison of the Zone 1 G values. FIG. 22 shows a side-by-side comparison of the Zone 1 B/G ratios. FIG. 23 shows a side-by-side comparison of the Zone 2 R values. FIG. 24 shows a side-by-side comparison of the Zone 2 G values. FIG. 25 shows a side-by-side comparison of the Zone 2 B values. FIG. 26 shows a side-by-side comparison of the Zone 2 B/G ratios. FIG. 27 shows a side-by-side comparison of the R values from Zone 4. FIG. 28 shows a side-by-side comparison of the B values from Zone 4. FIG. 29 shows a side-by-side comparison of the plots of the G values from Zone 4. FIG. 30 shows a side-by-side comparison of the plots of the B/G ratios from Zone 4. The three types of periorbital dyschromia herein may be distinguished from one another by analyzing the expression of certain genes, individually or collectively, in the dermis and/or epidermis of the periorbital dyschromic skin. The gene expression signature of a periorbital skin sample may be obtained by any suitable means known in the art. For example, genetic material may be obtained from a tissue sample provide by a donor (e.g., full thickness skin biopsy that exhibits a condition of interest) and subsequently processed using any suitable technology such as, for example, microarray analysis or NextGen sequencing. The gene expression signatures of the different types of periorbital dyschromia can then be compared to one another and/or a control to identify the differences in gene expression. Additionally or alternatively, it may be desirable to distinguish different types of periorbital dyschromia based on biological themes that correspond to the expression of certain genes, combinations of genes and/or gene families. Gene expression data may be analyzed by any suitable means known in the art.

Theme analysis may be used to identify biological or phenotypic themes associated with the gene expression data that correspond to Type I, Type II and Type III periorbital dyschromia. Theme analysis is a statistical analysis-based method for detecting biological patterns in gene expression data. The method uses an ontology of controlled vocabulary terms developed by the Gene Ontology (“GO”) Consortium [Ashburner, M. et al. (2000) Gene ontology: tool for the unification of biology. The Gene Ontology Consortium. Nat Genet, 25, 25-29] that describe the biological processes, molecular functions and cellular components associated with gene products. Analysis involves statistical comparison of a regulated list of genes and a larger reference list of all the expressed genes, to determine if genes annotated to specific GO terms are significantly enriched in the regulated list. This analysis may reveal biological patterns when multiple genes associated with a given GO term occur on the regulated list at a frequency greater than expected by chance. Such analysis may be performed using Theme Extractor proprietary software and an algorithm that calculates the p-value of each ontology term. Data may be analyzed for statistical significance, for example, by the Fisher's exact test. Conventional approaches and statistical methods such as, for example, Gene Set Enrichment Analysis described by Subramanian, A., et al., in “Gene set enrichment analysis: a knowledge-based approach for interpreting genome-wide expression profiles,” Proc. Natl. Acad Sci U.S.A, 102, 15545-155501 (2005) are suitable for conducting theme analysis herein.

### Biomarker

The three types of periorbital dyschromia herein may be distinguished from one another by collecting and analyzing the biomarkers present in periorbital skin. In particular, it has been found that the presence, absence and/or abundance of certain molecules in the epidermis of periorbital skin can be useful in distinguishing Type I, Type II and Type III periorbital dyschromia from one another. An example of such a molecule is pyrrole-2,3,5-tricarboxylic acid (“PTCA”), which is formed as a result of oxidative degradation of eumelanin. It has been found that Type I and Type III periorbital dyschromia have higher PTCA levels than Type II, and that Type I may exhibit higher PTCA levels than Type III, but not at a statistically significant amount. However, it is believed, without being limited by theory, that increasing the number of samples may cause this amount to become significant.

FIG. 13 illustrates the comparison between PTCA levels of samples obtained from: 1) a subject classified as No Dyschromia 382, 2) a subject who classified as Type I periorbital dyschromia 384, 3) a subject classified as Type II periorbital dyschromia 386, and 4) a test subject classified as Type III periorbital dyschromia 388. As illustrated in the chart 550 of FIG. 13, the PTCA levels of the Type I 384 and Type III 388 subjects were higher than the No Dyschromia 382 and the Type II subject 386 by a statistically significant amount. In contrast, the PTCA level of the Type II subject 386 was not higher than the No Dyschromia 382 by a statistically significant amount. PTCA levels herein are determined according to the Tape Stripping method described in U.S. patent application Ser. No. 13/957,278, filed Aug. 1, 2014, by Wehymeyer et al.

### Array of Products

Because different types of periorbital dyschromia appear to have different underlying, biological causes, it is important to provide an array of products tailored to treat the different types of periorbital dyschromia. In certain embodiments, the array may include two or more cosmetic products formulated to treat different types of periorbital dyschromia. For example, the array may include two or more cosmetic products wherein a first product in the array is formulated to treat Type I periorbital dyschromia and a second product is formulated to treat Type II periorbital dyschromia, with the second product containing an extract of Cucurbita pepo. In some embodiments, the array may include three products formulated to treat different types of periorbital dyschromia. For example, the array may include a first product formulated to treat Type I periorbital dyschromia, a second product is formulated to treat Type II periorbital dyschromia and a third product formulated to treat type III periorbital dyschromia, with the second and/or third products containing an extract of Cucurbita pepo. It is to be appreciated that each of the individual products may treat more than one type of periorbital dyschromia, as long as each product is formulated to treat at least one type of periorbital dyschromia that is different from at least one type of periorbital dyschromia treated by another product in the array. The products may be divided into any practical number of types based upon the number of specific types of periorbital dyschromia to be addressed by the product. For example, an array may be provided with two or more products which each correspond to one or more types of periorbital dyschromia, so long as one of the products contains an extract of Cucurbita pepo.

To help ensure that consumers are able to regularly and reproducibly identify and purchase the product in the array which is most appropriate to their type of periorbital dyschromia, it is further important to ensure that the product package quickly and effectively communicate the attributes and intended use of the products contained therein, while maintaining the common packaging elements of the product line-up. The common packaging element(s) may be used to identify a particular brand (e.g., brand name and/or trademark), and may further include indicia to quickly draw the consumer to the correct product to meet their periorbital dyschromia treatment need. Each product may include common indicia, which identifies the product as being part of the array (e.g., figure, color, name, shape), and product-specific indicia (e.g., one of the images shown FIG. 1, 2 or 3 or a product-specific word or description). In other words, the common indicia convey a message to the potential consumer that the products are from a particular manufacturer or source and have a common general use. The product-specific indicia convey a message to the potential consumer of a specific category of use under the general use for which the product is specialized.

The common indicia may be the same across all products in the array or may include common elements that allow a consumer to easily recognize that the products are part of the array. The product-specific indicia may vary across the products of the array. For example, a first package corresponding to a first type of periorbital dyschromia may have a first image of a periorbital region with a highlighted portion corresponding to the first type of periorbital. Continuing with this example, the array may include a second package corresponding to a second type of periorbital dyschromia which has a second image of a periorbital region with a highlighted portion corresponding to the second type of periorbital. In this example, the array may include a third package corresponding to a third type of periorbital dyschromia, which has a third image of a periorbital region with a highlighted portion corresponding to a third type of periorbital. All three packages in this example may include brand indicia and a common design indicia that assist in identifying the product contained by the package as being part of the product array from a particular manufacturer or supplier. Each of the first package, second package, and third package in this example may further include product-specific indicia, e.g., a word or picture description of the type of periorbital dyschromia associated with each package or a color indicia corresponding to the type of periorbital dyschromia associated with each package (e.g., colors associated with tanned skin, bruised skin or sunburned skin). Additionally, each package may include a representation of one or more product features such as applicators or regimens of use, which show how features of the products may vary across the array.

The products may be arranged at a retail environment within a single group of products separated into different types. For example, a group of products disposed upon a shelf within a “brick and mortar” retail environment, which are intended for use in treating periorbital dyschromia, may be divided based on the intended use of the products for treating two or more types of periorbital dyschromia (e.g., 3 types). In some embodiments, the products in the array may be spaced away from one another in a retail environment (e.g., different aisles or different areas of the same aisle), but should be positioned such that a consumer associates each product with the overall product line up (e.g., no more than 10 meters apart, 8 meters apart, 5 meters apart, 3 meters apart, 1 meter apart, or adjacent one another on the same shelf). Examples of approaches for providing arrays of products which intended for use in a specific category but under the general use for which the product is specialized are described in U.S. Pat. Nos. 6,648,864 and 6,763,944 and U.S. Publication No. 2006/0193898. These disclosures provide additional detail regarding characteristics and methods of tailoring products to different product-specific uses and communicating such tailoring.

In certain embodiments, the retail environment may be a virtual retail environment (e.g., an on-line store where consumers purchase goods over the internet). The array of products should be arranged in the virtual retail environment such that a consumer can associate an individual product in the array to the overall product line up.

Each product in the present array may include a composition containing one or more materials suitable for addressing one or more characteristics associated with a type of periorbital dyschromia. Examples of such characteristics include, without limitation, visual indicators such as the location of the periorbital dyschromia in the periorbital region, imaging indicators such as RGB values, RBX® values or L*a*b*C*h* values, histological indicators such as the presence or abundance of Fontanna-Masson positive bodies in the dermis of periorbital skin tissue, biomarker characteristics such as the presence or amount of pyrrole-2,3,5-tricarboxylic acid present in periorbital skin tissue (e.g., determined according to a tape stripping method), and genetic indicators such as reversing undesirable gene expression (e.g., determined using an in vitro or ex vivo analysis) and/or reversing gene expression signatures (e.g., determined using connectivity mapping). For example, the array may include two or more products that each includes a composition for reversing the expression of one or more genes associated with different types of periorbital dyschromia. In a particularly suitable example, the array may include a first product composition that includes one or more ingredients for addressing a characteristic associated with Type I periorbital dyschromia (e.g., reversing the expression of one or more genes from Tables 1 to 4 or one or more of the genes associated with the biological pathways shown in Tables 13 and 16, as disclosed in U.S. patent application Ser. No. 14/472,716) and a second product composition that includes one or more ingredients for addressing a characteristic associated with Type II periorbital (e.g., reversing the expression of one or more genes from table 5 to 8 or one or more of the genes associated with the biological pathways shown in tables 14 and 17, as disclosed in U.S. patent application Ser. No. 14/472,716). Continuing with this example, in certain embodiments, the array may include a third product composition that includes one or more ingredients for addressing a characteristic associated with Type III periorbital (e.g., reversing the expression of one or more genes from table 9 to 12 or one or more of the genes associated with the biological pathways shown in tables 15 and 18, as disclosed in U.S. patent application Ser. No. 14/472,716). For these examples, either or both of the second and third products will include a composition comprising extracts Cucurbita pepo.

Nonlimiting examples of agents that may be suitable for addressing a characteristic associated with Type I periorbital dyschromia are listed in table 19 of U.S. patent application Ser. No. 14/472,716. Nonlimiting examples of agents that may be suitable for addressing a characteristic associated with Type II periorbital dyschromia are listed in table 20 of U.S. patent application Ser. No. 14/472,716. Nonlimiting examples of agents that may be suitable for addressing a characteristic associated with Type III periorbital dyschromia are listed in table 21 of U.S. patent application Ser. No. 14/472,716.

In some embodiments, one or more of the products in the array may be provided by placing in a container suitable for containing the product in a stable form and enabling access to the product by a user. The products herein may be disposed in any suitable container known in the art for storing cosmetic products of the type disclosed herein. For example, the container may be in the form of a gas impermeable jar, which prevents volatile material from escaping and provides an opening that enables a user to access the product. In this example, a user may transfer product from the jar to a target skin area using one or more fingers. In some embodiments, the product may be applied to a target skin area using a suitable implement.

In one embodiment, the array of products will include a product (e.g., the second product) that provides a decrease in Laser Speckle Value in Type II periorbital dyschromia. In one embodiment, the array will include a product (e.g., the third product) that provides an improvement in Visual Perception Scale in Type III periorbital dyschromia. In one embodiment, the array will include a product that reduces melanin production as measured in the BC 16 melanin inhibition assay. In one embodiment, the array will include a product (e.g., the first product) that is a component of a collection of products marketed to provide users with periorbital skin benefits.

### Kits

In some embodiments, it may be desirable to market two or more products of the array described above as a kit. That is, the products are arranged in a common package and marketed or sold as a unitary product offering. The products in the kit may include individual packaging, which is substantially the same as or different from the packaging of the individual products.

### Methods of Use

U.S. Provisional App. No. 61/798,340, filed by Osorio, et al., on Mar. 15, 2013 and titled “Method of Treating Periorbital Dyschromia” discloses suitable methods of using the products, arrays and kits described herein for treating different types of periorbital dyschromia.

### Compositions

A. Cucurbita pepo Active

The compositions for use in the array of products herein include an effective amount of Cucurbita pepo (pumpkin seed) extract disposed in a dermatologically acceptable carrier. The pumpkin seed extract can be obtained using any of a variety of known extraction techniques. An example of a pumpkin seed extract suitable for use herein is OCALINE PF, available from Soliance US, Allendale, N.J. (a subsidiary of Givaudan). OCALINE PF is a blend of sea water and pumpkin seed vegetable.

The pumpkin seed extract can be included in a topical cosmetic composition as a chronic active for treating periorbital dyschromia, in particular Type II and/or Type III periorbital dyschromia. The pumpkin seed extract may be present at from 0.0001% to 15%, from 0.0002% to 10%, from 0.001% to 15%, from 0.025% to 10%, from 0.05% to 10%, from 0.05% to 5%, or even from 0.1% to 5%, by weight of the total composition. The amount of pumpkin seed extract that is “effective” can differ from one particular source (e.g., manufacturer) of extract to another, and can be determined by the skilled artisan based upon the particular extract product's level of activity (e.g., level of active components present). As with any extract, the concentration of active components in the particular extract product to be used will depend on factors such as the final dilution volume of the extract product, the particular extraction method employed, the natural range of variation among individual plants, and other common factors known to those skilled in the art.

B. Optional Agents

In some embodiments, it may be desirable to include one or more skin care agents in the composition(s) (e.g., skin tone agent(s), anti-aging agents(s), anti-inflammatory agent(s), sunscreen agent(s)). The skin care agent may optionally be a chronic active. For example, one or more skin tone agents may be included to further improve overall skin tone. When present, the compositions of the present invention may contain up to about 50%, 40%, 30%, 20%, 10%, 5%, or 3%, by weight of the composition, of a skin-care active. When present, the compositions of the present invention contain at least about 0.001%, 0.01%, 0.1%, 0.2%, 0.5%, or 1%, by weight of the composition, of the skin tone agent. Suitable ranges include any combination of the lower and upper limits including suitable ranges from about 0.1% to about 50%; from about 0.2% to about 20%; or from about 1% to about 10%, by weight of the composition, of the skin tone agent. The amounts listed herein are only to be used as a guide, as the optimum amount of the skin-care active will depend on the specific active selected since their potency does vary considerably. Nonlimiting examples of ingredients commonly used in cosmetic compositions, skin-care actives, methods of identifying skin-care actives and/or methods of formulating skin care compositions are described in U.S. Publications Nos. US2002/0022040; US2003/0049212; US2007/0196344; US2008/0181956; US2010/00092408; US2008/0206373; US 2010/0239510; US2010/0189669; US2011/0262025; US2011/0097286; US2012/0197016; US2012/0128683; US2012/0148515; US2012/0156146; and US2013/0022557 and U.S. Pat. Nos. 5,939,082; 5,872,112; 6,492,326; 6,696,049; 6,524,598; 5,972,359; and 6,174,533.

Suitable skin tone agents include, but are not limited to, sugar amines, vitamin B₃ compounds, arbutin, deoxyarbutin, 1,3-dihydroxy-4-alkylbenzene such as hexylresorcinol, sucrose dilaurante, bakuchoil (4-[(1E,3S)-3-ethenyl-3,7-dimethyl-1,6 octadienyl] phenol or monterpene phenol), pyrenoine (available from Biotech Marine, France), panicum miliaceum seed extract, arlatone dioic acid, cinnamic acid, ferulic acid, achromaxyl, methyl nicotinamide, oil soluble licorice extract, folic acid, undecylenic acid (i.e., undecenoic acid), zinc undecylenate, thiamine (Vitamin B1) and its hydrochloride, L-tryptophan, helianthus annuus (sunflower) and vitis vinifera (grape) leaf extract, carnosine (i.e., dragosine), methyl gentisate, 1,2-hexandiol and 1,2-octandiol (i.e., combination sold as Symdiol 68 by Symrise A G, Germany), inositol, undecylenoyl phenylalanine (e.g., sold under the tradename Sepiwhite by Seppic, France), kojic acid, hexamidine compounds, salicylic acid, and retinoids including retinol and retinyl propionate.

Suitable anti-aging agents include, but are not limited to, sulfur-containing D and L amino acids and their derivatives and salts, particularly the N-acetyl derivatives, a (e.g., N-acetyl-L-cysteine); thiols (e.g. ethane thiol); hydroxy acids (e.g., alpha-hydroxy acids such as lactic acid and glycolic acid and beta-hydroxy acids such as salicylic acid and salicylic acid derivatives such as the octanoyl derivative); phytic acid, lipoic acid; lysophosphatidic acid, skin peel agents (e.g., phenol and the like), vitamin B₃ compounds and retinoids.

The composition(s) can additionally comprise anti-inflammatory agents, which can be useful for improving the appearance of perceived skin tone flaws resulting from skin inflammation. Transient inflammatory events triggering hyperpigmentation and, more specifically, post-inflammatory hyperpigmentation include, but are not limited to, acne lesions, ingrown hairs, scratches, insect bites, surfactant damage, allergens, and short-term UV exposure. Inflammation induced hyperpigmentation including post-inflammatory hyperpigmentation may be managed by incorporating into the compositions of the present invention an anti-inflammatory agent. When present, the compositions of the present invention contain up to about 20%, 10%, 5%, 3%, or 1% by weight of the composition, of the anti-inflammatory agent. When present, the compositions of the present invention contain at least about 0.001%, 0.01%, 0.1%, 0.2%, 0.3%, 0.5%, or 1%, by weight of the composition, of the anti-inflammatory agent. Suitable ranges include any combination of the lower and upper limits. Suitable anti-inflammatory agents include, but are not limited to nonsteroidal anti-inflammatory agents (NSAIDS including but not limited to ibuprofen, naproxen, flufenamic acid, etofenamate, aspirin, mefenamic acid, meclofenamic acid, piroxicam and felbinac), glycyrrhizic acid (also known as glycyrrhizin, glycyrrhixinic acid, and glycyrrhetinic acid glycoside) and salts such as dipotassium glycyrrhizate, glycyrrhetenic acid, licorice extracts, olive oil extracts (e.g., Olivem®), bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, epigallocatechin gallatyl glucoside, chenopodium Quinoa Seed Extract, propyl gallate, gallyl glucoside, epigallocatechin gallatyl glucoside, red clover extract, and sea whip extract (extracts from plant in the order Gorgonacea), derivatives of any of the foregoing, and mixtures thereof.

The compositions may comprise one or more sunscreen agents and/or ultraviolet light absorbers. Herein, “sunscreen agent” includes, ultraviolet light absorbers and physical sunblocks. Sunscreen agents may be organic or inorganic. Examples of suitable sunscreen agents are disclosed in Personal Care Product Council's International Cosmetic Ingredient Dictionary and Handbook, Thirteenth Edition, as “sunscreen agents.” Particularly suitable sunscreen agents are 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

In one embodiment, the composition may comprise from about 1% to about 20%, and alternatively from about 2% to about 10% by weight of the composition, of the sunscreen agent. Exact amounts will vary depending upon the chosen sunscreen agent and the desired Sun Protection Factor (SPF), which is within the knowledge of one of skilled in the art.

In one embodiment, a composition (e.g., a composition for treating Type II or Type III periorbital dyschromia) may comprise Vicia faba, sometimes referred to as fava bean or broad bean extract. Fava bean extract has the INCI name: Vicia Faba Seed Extract; CAS No. 89958-06-5.

C. Other Optional Components

The compositions may contain a variety of other ingredients provided that they do not unacceptably alter the benefits of the invention. When present, compositions of the present invention may contain from about 0.0001% to about 50%; from about 0.001% to about 20%; or, alternately, from about 0.01% to about 10%, by weight of the composition, of the optional components. The amounts listed herein are only to be used as a guide, as the optimum amount of the optional components used in a composition will depend on the specific active selected since their potency does vary considerably. Hence, the amount of some optional components useful in the present invention may be outside the ranges listed herein.

The optional components, when incorporated into the composition, should be suitable for use in contact with human skin tissue without undue toxicity, incompatibility, instability, allergic response, and the like. The compositions of the present invention may include optional components such as anti-acne actives, desquamation actives, anti-cellulite agents, chelating agents, flavonoids, tanning active, non-vitamin antioxidants and radical scavengers (e.g., amino acids essential for hemoglobin formation, L-leucine, Pitera, Haloxyl), hair growth regulators, anti-wrinkle actives, anti-atrophy actives, minerals, phytosterols and/or plant hormones, N-acyl amino acid compounds, antimicrobial or antifungal actives, and other useful skin care actives, which are described in further detail in U.S. Publication Nos. US 2006/0275237 and US 2004/0175347.

The Personal Care Product Council's International Cosmetic Ingredient Dictionary and Handbook, Thirteenth Edition, describes a wide variety of non-limiting cosmetic and pharmaceutical ingredients commonly used in the skin care industry, which are suitable optional components for use in the compositions of the present invention. Examples of these ingredient classes include: abrasives, absorbents, aesthetic components such as fragrances, pigments, colorings/colorants, essential oils, anti-caking agents, antifoaming agents, antimicrobials, binders, biological additives, buffering agents, bulking agents, chelating agents, chemical additives, colorants, cosmetic astringents, cosmetic biocides, denaturants, drug astringents, emollients, external analgesics, film formers or materials, opacifying agents, pH adjusters, preservatives, propellants, reducing agents, sequestrants, skin cooling agents, skin protectants, thickeners viscosity modifiers, vitamins, and combinations thereof.

D. Optional Dermatologically Acceptable Carrier

The compositions may also comprise a dermatologically acceptable carrier (which may be referred to as “carrier”) for the composition. The phrase “dermatologically acceptable carrier”, as used herein, means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

The aqueous phase typically comprises water. However, in other embodiments, the aqueous phase may comprise components other than water, including but not limited to water-soluble moisturizing agents, conditioning agents, anti-microbials, humectants and/or other water-soluble skin care actives. In one embodiment, the non-water component of the composition comprises a humectant such as glycerin and/or other polyols. However, it should be recognized that the composition may be substantially (i.e., less than 1% water) or fully anhydrous.

A suitable carrier is selected to yield a desired product form. Furthermore, the solubility or dispersibility of the components (e.g., extracts, sunscreen active, additional components) may dictate the form and character of the carrier. In one embodiment, an oil-in-water or water-in-oil emulsion is preferred.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

### METHODS

### Imaging

This method provides a suitable means for capturing a reproducible and analyzable image. Any suitable image capture device along with imaging software and other associated ancillary equipment (e.g., computer and lights) may be used. A particularly suitable imaging system is the Visia-CR® brand imaging system, available from Canfield Scientific, New Jersey. The Visia® brand imaging system incorporates a Canon® brand EOS-1Ds Mk III SLR camera, which includes a CMOS sensor and provides 21.1 Mega pixel resolution (14-bit A/D converter).

Images may be collected under different lighting modalities using standard light, UV, cross-polarization, parallel-polarization or a combination of these. For example, the values and ranges described herein are reported using a (D65/2) light source. One skilled in the art will appreciate that these values can be reported at a wide range of different illuminations (D50, D75, Illuminant A, F2, F7, F11, TL84, etc. or 2 or 10 degree observer) according to well known conversion methods, and when such conversions occur, the color values will typically change accordingly. In other words, even though the actual limits and/or ranges may change based on the conditions under which the image is captured, similar relationships among the values and ranges will still be seen. For example, if the camera has lower spectral sensitivity in the red channel than the camera described herein, the R channel response may be lower and the corresponding L*a*b*C*h* (“LabCh”) color values will be different, which in this case may result in lower a* values, higher b* values, and/or higher hues. Accordingly different camera sensitivities, lightings and relevant exposures are contemplated by the method herein, and the actual limits and/or ranges disclosed herein may vary according to the particular circumstances in which the image is captured without departing from the scope of the systems and/or methods described herein.

In preparation for image capture, test subjects are required to wash their faces and wait for at least 15 minutes to let their face dry. The hair of the subject is covered with a hairnet and the head and shoulders of the subject are covered with a black cloth. All jewelry that can be seen in an image area of interest is removed. The subject is positioned such that the subject's chin is resting comfortably on the chin rest of the imaging system, and a front image of the face (as opposed to a left-side or right-side image) can be suitably captured by the image capture device. After the subject is positioned, one or more images are captured (e.g., between 1 and 24, 2 and 20 or even between 3 and 15) with the subject's eyes open. It can be important to ensure that the subject's eyes are open when the image is captured, otherwise the closed upper eyelid may cause an inaccurate pigmentation reading. The captured image(s) are processed by converting the raw image to a .jpg file format.

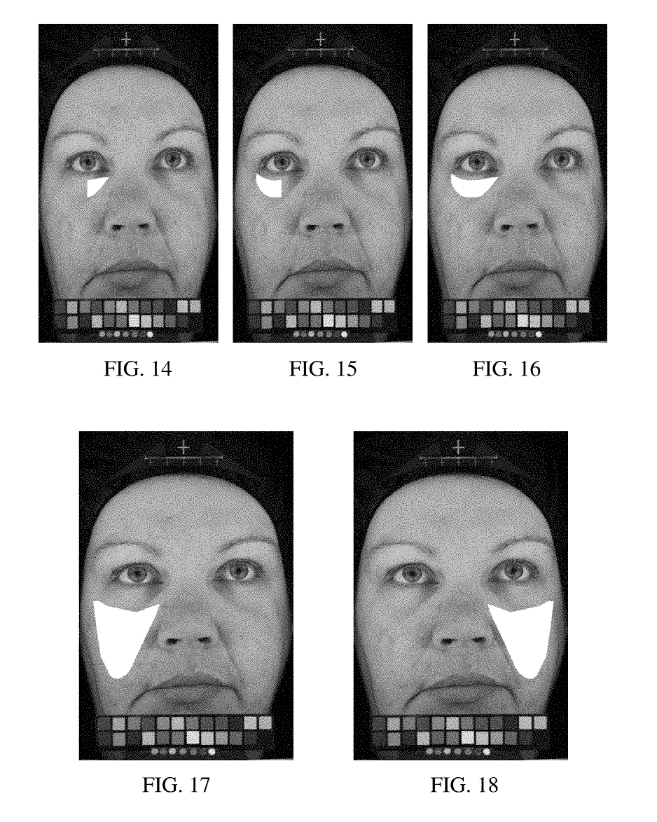

Next, the .jpg format image is analyzed by a computer with suitable image analysis software. In some instances, it may be desirable to analyze only a portion of the image (e.g., Zone 1, 2 and/or 3 of the periorbital region). The portion of the image to be analyzed may be “masked,” for example, as shown in FIGS. 14 to 18, using image editing software such as Photoshop® or ImageJ®. The masked region can then be isolated and analyzed as a separate image. FIG. 14 illustrates an example of a masked region corresponding to Zone 1, which is referred to herein as “Mask A.” FIG. 15 illustrates an example of a masked region corresponding to Zone 2, which is referred to herein as “Mask B.” FIG. 16 illustrates an example of a masked region corresponding to Zones 1 and 2, which is referred to herein as “Mask C.” FIGS. 17 and 18 both illustrate an example of a masked region corresponding to the cheek. It is to be appreciated that the image need not necessarily be masked for suitable analysis, and in some instances the entire image may be analyzed. In some instances, it may be desirable to reduce the size of the image, mask and/or region of interest by several pixels (e.g., between 5 and 15 pixels) around the outer edge of the image where some shadowing may occur.

In some instances, it may be desirable to convert some or all of the RGB values in the image or a portion thereof to LabCh values. The LabCh values can be calculated using a suitable RGB conversion tool at D65 Illuminant and 2 degree observer (i.e., D65/2). This conversion can be performed by software installed on the computer or a suitable conversion tool may be found online, for example, at: http://www.easyrgb.com/index.php?X=CALC). The conversion from RGB values to LabCh values can be performed on the entire image, a portion thereof or on one or more individual pixels. The resulting LabCh values may be averaged to provide average values for the image, mask or region of interest.

In some instances, the pixels may be analyzed individually and each pixel classified as corresponding to a particular type of periorbital dyschromia based on one or more of the LabCh values. When analyzed individually, the pixels may be analyzed according to their distribution across the different types of periorbital dyschromia. An example of classifying periorbital dyschromia based on individual pixel distributions is described below in Example 2. Since color may be perceived as being relative, depending on, for example, which instruments and/or imaging system is used, it can be important to color correct the masked region for each subject using a suitable color correction technique (e.g., according to International Color Consortium standards and practices), which helps make the color determination by the system less instrument specific. In some instances, it may be desirable to normalize the color in a region of interest (e.g., a masked region) to the basal skin tone of a nearby region (e.g., cheek). For example, the basal skin tone of the cheek may be obtained by masking a region of interest in the cheek (e.g., as illustrated in FIG. 20 or 21) and converting the RGB values in the masked region to L*a*b*C*h* values as described above. The resulting basal skin tone values for the cheek may then be subtracted from the corresponding values in the region of interest to provide normalized values. Color normalization may be performed on the entire region of interest (e.g., an average value for the ROI) or on a pixel by pixel basis for some or all of the pixels in the ROI, which may be 200,000 or more pixels. The following formula provides an illustration of color normalization.

DL1=Lpixell=Lcheekave

DC1=Cpixell−Ccheekave

Dh1=hpixell−hcheek ave

Where DL, DC and Dh are the normalized L*, C*, and h* values, respectively, of a pixel in the mask region; Lpixel, Cpixel and hpixel are the respective L*, C* and h* values of the pixels as determined from the image analysis; and Lcheekave, Ccheekave and hcheekave are the respective average L*, C* and h* values determined for the cheek.

### Visual Perception Scale

This method provides a way to quantitatively evaluate the change in appearance of periorbital dyschromia using a Visual Perception Scale (“VPS”). The visual grading described herein is conducted by trained graders on captured images of the test subjects, but the method may also be readily adapted for use by consumers in self-diagnosing periorbital dyschromia and/or by in vivo examination of the periorbital region of a person by another. The images used in this method may be captured using the Imaging Method described in more detail below. Comparisons of baseline images collected at week 0 versus subsequent time point images are performed. The degree of change is scored using a −4 to +4 Magnitude Scale as shown below in Table D. Negative numbers indicate that the periorbital dyschromia appeared better at baseline, while positive numbers reflect an improvement of the subject's appearance relative to baseline. The area of the periorbital region graded encompasses the area of the eye socket generally under the eye, extending from the inner corner of the eye, along the cheek bone and around to the outer corner of the eye, inclusive of the lateral orbital rim. The area of the periorbital region graded in this method does not include the area directly below the lower eyelid (as demarcated by the lower eyelashes), the upper eyelid or the upper eye socket. Features considered by the graders include: 1) the relative appearance of the darkness of the discoloration of the periorbital dyschromia compared to the surrounding skin tone; 2) the amount of affected area, footprint or pattern of the periorbital dyschromia; and 3) the appearance of the pigmentation hues involved in the discoloration and their intensity.

TABLE D
Magnitude Scale

| Grade | Anchor | Description |
|---|---|---|
| +4 | Outstand- | Significant improvement in contrast, area |
| | ingly | and/or intensity throughout the graded area; |
| | Improved | outstanding improvement is immediately seen. |
| +3 | Obviously | Readily seen improvements in contrast, area |
| | Improved | and/or intensity are obvious almost instantly. |
| +2 | Visibly | Visible improvement in contrast, area and/or |
| | Improved | intensity is able to be seen within a few seconds. |
| +1 | Perceptibly | Improvement in contrast, area and/or intensity |
| | Improved | are perceived after careful study. |
| 0 | Neutral/No | No changes, or equivalent positive and negative |
| | Difference | changes, in the graded area.* |
| | | * Images should not be over scrutinized; images |
| | | requiring more than 30 seconds of study to |
| | | identify a change should be scored as having |
| | | “zero” change. |
| −1 | Perceptibly | Worsening in contrast, area and/or intensity are |
| | Worsened | perceived after careful study. |
| −2 | Visibly | Visible worsening in contrast, area and/or |
| | Worsened | intensity is able to be seen within a few seconds. |
| −3 | Obviously | Readily seen worsening in contrast, area and/or |
| | Worsened | intensity are obvious almost instantly. |
| −4 | Outstand- | Significant worsening in contrast, area and/or |
| | ingly | intensity throughout the graded area; |
| | Worsened | outstanding improvement is immediately seen. |

### Blood Perfusion

Blood perfusion is generally recognized as the process of delivering blood to a capillary bed in biological tissue. Blood vessels and blood in the capillary beds of the periorbital region may be visible through the relatively thin periorbital skin. Thus, when less blood is visible in and around the capillary beds of the periorbital skin, there is a corresponding improvement in the appearance of periorbital dyschromia. The Blood Perfusion method provides a suitable method of measuring the change in the amount of blood present in the capillary beds of periorbital skin. The Blood Perfusion method uses a blood perfusion imager (e.g., PeriCam™ PSI brand imager or equivalent), which is based on Laser Speckle Contrast Analysis (“LASCA”) technology, in conjunction with PIMsoft™ brand dedicated application software or equivalent to visualize tissue blood perfusion in real-time. Test subjects are comfortably seated within 10 to 25 cm of the imager and instructed to close their eyes. Three images (i.e., perfusion, intensity and a standard color image) of the test subject's face are captured and recorded by the imager in accordance with the manufacturer's instructions. Using the dedicated application software, the periorbital regions of the test subject are masked (i.e., designated as regions of interest) to obtain the perfusion measurement in a periorbital region of interest. Masking is described in more detail below in the Imaging Method.

### EXAMPLES

Table 1 shows five exemplary oil-in-water emulsion cosmetic compositions for use in the present arrays. Compositions A to E may be prepared as follows. Combine the water phase ingredients in a suitable vessel and heat to 75° C. In a separate suitable vessel, combine the oil phase ingredients and heat to 75° C. Add the oil phase to the water phase and mill the resulting emulsion (e.g., with a TEKMAR™ T-25 or equivalent). Add the thickener to the emulsion and cool to 45° C. while stirring. At 45° C., add the remaining ingredients. Cool the product with stirring to 30° C. and pour into suitable containers.

TABLE 1

| | A | B | C | D | E |
|---|---|---|---|---|---|
| Water Phase: | | | | | |
| Water | qs | qs | qs | qs | qs |
| Glycerin | 3.0 | 5.0 | 7.0 | 10.0 | 15.0 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2.0 | 0.5 | 3.5 | 3.0 | 5.0 |
| D-panthenol | 0.5 | 0.1 | 1.0 | 0.5 | 1.5 |
| Sodium Hydroxide | 0.001 | 0.002 | 0.001 | 0.001 | 0.001 |
| Benzyl alcohol | 0.25 | 0.25 | 0.25 | 0.25 | 0.25 |
| FD&C Red #40 | — | — | — | 0.0005 | — |
| Ocaline⁴ | 1.0 | 15.0 | 0.1 | 5.0 | 2.0 |
| Palmitoyl-pentapeptide¹ | 0.0002 | — | — | — | 0.0003 |
| N-acetyl glucosamine | 2.0 | — | 2.0 | — | 5.0 |
| Oil Phase: | | | | | |
| Isohexadecane | 3.0 | 3.0 | 3.0 | 4.0 | 3.0 |
| Isopropyl Isostearate | 1.0 | 0.5 | 1.3 | 1.5 | 1.3 |
| Sucrose polyester | 0.7 | — | 0.7 | 1.0 | 0.7 |
| Octinoxate | — | — | — | — | 6.0 |
| Avobenzone | — | — | — | 2.0 | 0.5 |
| Ethylhexyl methoxycrylene | — | — | — | — | 0.5 |
| Homosalate | — | — | — | 4.0 | — |
| Octisalate | — | — | — | 4.0 | — |
| Octocrylene | — | — | — | 2.0 | — |
| Phytosterol | — | — | — | 0.1 | — |
| Cetyl alcohol | 0.4 | 0.3 | 1.0 | 0.5 | 0.4 |
| Stearyl alcohol | 0.5 | 0.35 | 1.0 | 0.6 | 0.5 |
| Behenyl alcohol | 0.4 | 0.3 | 1.0 | 0.5 | 0.4 |
| PEG-100 stearate | 0.1 | 0.1 | 0.1 | 0.2 | 0.1 |
| Stearic Acid | 0.1 | 0.05 | 0.1 | 0.2 | 0.1 |
| Cetearyl glucoside | 0.1 | 0.1 | 0.1 | 0.25 | 0.1 |
| Thickener: | | | | | |
| Polyacrylamide/C13-14 isoparaffin/ | 1.5 | — | 2.0 | 2.5 | 2.0 |
| laureth-7 | | | | | |
| Sodium acrylate/sodium | — | 3.0 | — | — | — |
| acryloyldimethyl taurate copolymer/ | | | | | |
| isohexadecane/polysorbate 80 | | | | | |
| Additional Ingredients: | | | | | |
| KTZ Interfine ™ Gold² | 2.5 | — | 0.3 | — | 0.5 |
| KTZ Interfine ™ Red² | — | 1.0 | — | — | 0.5 |
| Tapioca Starch | — | 5.0 | — | 2.0 | 0.5 |
| Dry Flo TS³ | 8.0 | — | 1.5 | — | — |
| Dimethicone/dimethiconol | — | 1.0 | 2.0 | 0.5 | 2.0 |
| Fragrance | — | 0.1 | 0.1 | 0.1 | 0.1 |
| Polymethylsilsequioxane | — | — | 0.25 | — | 1.0 |
| Nylon-12 | — | 0.5 | — | — | — |
| Total: | 100% | 100% | 100% | 100% | 100% |

¹Palmitoyl-lysine-threonine-threonine-lysine-serine available from Sederma (France)
²Titanium dioxide coated mica available from Kobo Products Inc.
³Tapioca starch and polymethylsilsesquioxane from Akzo Nobel
⁴Available from Soliance US, Allendale, New Jersey (a subsidiary of Givaudan)

Table 2 shows five exemplary silicone-in-water emulsion cosmetic compositions for use in the present arrays. Compositions F to J may be prepared as follows. In a suitable vessel, combine the water phase ingredients and mix until uniform. In a separate suitable container, combine the silicone/oil phase ingredients and mix until uniform. Add half the thickener and then the silicone/oil phase to the water phase and mill the resulting emulsion (e.g., with a Tekmar™ T-25). Add the remainder of the thickener and then the remaining ingredients to the emulsion while stirring. Once the composition is uniform, pour the product into suitable containers.

TABLE 2

| | F | G | H | I | J |
|---|---|---|---|---|---|
| Water Phase: | | | | | |
| Water | qs | qs | qs | qs | qs |
| Glycerin | 3.0 | 5.0 | 7.0 | 10.0 | 15.0 |
| Disodium EDTA | 0.1 | 0.1 | 0.05 | 0.1 | 0.1 |
| Niacinamide | 2.0 | 0.5 | 3.5 | 3.0 | 5.0 |
| D-panthenol | 0.5 | 0.1 | 1.0 | 0.5 | 1.5 |
| Ocaline⁹ | 1.0 | 2.0 | 0.1 | 10.5 | 1.5 |
| FD&C Yellow #10 | — | — | — | — | 0.0004 |
| Palmitoyl-pentapeptide¹ | 0.0002 | — | — | — | 0.0003 |
| N-acetyl glucosamine | 2.0 | — | 2.0 | — | 5.0 |
| Silicone/Oil Phase: | | | | | |
| Cyclomethicone D5 | 10.0 | 5.0 | 5.0 | 10.0 | 7.5 |
| Dow Corning ® 9040 silicone | — | 10.0 | 5.0 | 5.0 | 7.5 |
| elastomer² | | | | | |
| KSG-15AP silicone Elastomer³ | 5.0 | — | 5.0 | 5.0 | 7.5 |
| Dimethione/dimethiconol | — | 2.0 | 2.0 | 1.0 | 2.0 |
| Dimethicone 50 csk | 1.0 | — | — | — | — |
| Laureth-4 | 0.2 | 0.2 | 0.3 | 0.2 | 0.2 |
| Vitamin E Acetate | — | 0.5 | — | 0.1 | — |
| Thickener: | | | | | |
| Polyacrylamide/C13-14 isoparaffin/ | 2.5 | 2.5 | 3.0 | — | — |
| laureth-7 | | | | | |
| Sodium acrylate/sodium | — | — | — | 3.0 | — |
| acryloyldimethyl taurate copolymer/ | | | | | |
| isohexadecane/polysorbate 80 | | | | | |
| Acrylates/C10-30 alkyl acrylates | — | — | — | — | 0.5 |
| crosspolymer | | | | | |
| Additional Ingredients: | | | | | |
| KSP 100⁴ | 6.0 | 1.5 | — | — | — |
| KTZ Interval ™ Green⁵ | — | 0.35 | — | 1.0 | 0.8 |
| Prestige Silk ™ Blue⁶ | — | — | 1.5 | — | — |
| Cosmica ™ Orange⁷ | — | — | — | 0.1 | — |
| Dry Flo TS⁸ | — | 1.5 | 8.0 | — | — |
| Fragrance | — | 0.1 | 0.1 | 0.1 | 0.1 |
| Triethanolamine | — | — | — | — | 0.6 |
| PTFE | — | 0.5 | — | — | — |
| Polymethylsilsequioxane | — | 0.5 | 1.0 | — | — |
| Polyethylene | — | 0.5 | — | — | 1.0 |
| Total: | 100% | 100% | 100% | 100% | 100% |

¹Palmitoyl-lysine-threonine-threonine-lysine-serine available from Sederma (France)
²A silicone elastomer dispersion from Dow Corning Corp.
³A silicone elastomer dispersion from Shin Etsu
⁴Vinyl dimethicone/methicone silsesquioxane crosspolymer from Shinetsu
⁵Titanium dioxide coated mica from Kobo Products Inc.
⁶Titanium dioxide and tin oxide coated mica from Eckart.
⁷Iron oxide coated mica from Engelhard Corporation.
⁸Tapioca starch and polymethylsilsesquioxane from Akzo Nobel
⁹Available from Soliance US, Allendale, New Jersey

Table 3 shows two exemplary water-in-silicone emulsion cosmetic compositions for use in the present arrays. Compositions K and L may be prepared as follows. In a suitable vessel, blend the Phase A components with a suitable mixer until all of the components are dissolved. Blend Phase B components in suitable vessel and mix until uniform. Add Phase A slowly to Phase B with mixing and continue mixing until uniform. Mill the resulting product for about 5 minutes using an appropriate mill (e.g., TEKMAR T-25). Next, add Phase C while stirring the product. Continue mixing until the product is uniform, and pour the product into suitable containers.

TABLE 3

| | K | L |
|---|---|---|
| Phase A | | |
| Water | q.s. | q.s. |
| Allantoin | 0.2 | 0.2 |
| Disodium EDTA | 0.1 | 0.1 |
| Ethyl paraben | 0.2 | 0.2 |
| Propyl paraben | 0.1 | 0.1 |
| BHT | 0.015 | 0.015 |
| D-panthenol | 1.0 | 0.5 |
| Glycerin | 7.5 | 13.0 |
| Niacinamide | 2.0 | 3.5 |
| Palmitoyl-pentapeptide¹ | — | 0.0003 |
| Benzyl alcohol | 0.2500 | 0.2500 |
| Green tea extract | 1.0 | 0.1 |
| Ocaline⁶ | 5.0 | 2.2 |
| Sodium metabisulfite | 0.1 | 0.01 |
| Phase B | | |
| Cyclopentasiloxane | 15.0000 | 15.0000 |
| C12-C15 alkyl benzoate | 1.5 | — |
| Vitamin E acetate | 0.5 | 0.1 |
| Retinyl propionate | 0.15 | — |
| Phytosterol | 0.1 | — |
| KSG-21 silicone elastomer² | 4.0 | 4.0 |
| Dow Corning ® 9040 silicone | 15.0 | 15.0 |
| elastomer³ | | |
| Abil ™ EM-97 dimethicone | 0.5 | — |
| copolyol⁴ | | |
| Polymethylsilsesquioxane | 2.5 | 0.5 |
| Fragrance | — | 0.1 |
| Phase C | | |
| KTZ Interval ™ Red-1152⁵ | — | 0.3 |

¹Palmitoyl-lysine-threonine-threonine-lysine-serine available from Sederma (France)
²KSG-21 is an emulsifying silicone elastomer available from Shin Etsu
³A silicone elastomer dispersion from Dow Corning Corp
⁴Abil EM-97 available from Goldschmidt Chemical Corporation
⁵Silane surface treated titanium dioxide coated mica from Koho Products Inc.
⁶Available from Soliance US, Allendale, New Jersey

Table 4 shows examples of personal care compositions for use in the present method. The compositions are prepared by first combining the water phase ingredients in a container and mixing while heating to ˜75° C. until uniform. Meanwhile, the ingredients of part 1 of the oil phase are weighed into a separate container and mixed while heating to ˜75° C. until uniform. Once both respective phases are uniform, part 1 of the oil phase is added to the water phase. The resulting mixture is subjected to high shear mixing (e.g., Flacktek Speedmixer, or rotor-stator mill) and then cooled while stirring. When the temperature reaches ˜60° C., the thickener is then added while continuing to stir. Finally, when the batch reaches ˜50° C., the Oil Phase Part 2 is added; ingredients are added individually as cooling continues. At ˜40° C., the active (i.e., pumpkin seed extract) is added while stirring. Once all ingredients are in the formulation and the temperature is ˜40° C., the resulting mixture is again subjected to high shear mixing, and then the product is poured into suitable containers.

TABLE 4

| | M | N | O | P | Q | R |
|---|---|---|---|---|---|---|
| Water Phase: | | | | | | |
| Water | 84.26 | 84.22 | 84.17 | 84.02 | 83.77 | 79.27 |
| Glycerin | 5.0 | 5.0 | 5.0 | 5.0 | 5.0 | 5.0 |
| Disodium EDTA | 0.1 | 0.1 | 0.1 | 0.1 | 0.1 | 0.1 |
| Oil Phase Part 1: | | | | | | |
| Isohexadecane | 3.0 | 3.0 | 3.0 | 3.0 | 3.0 | 3.0 |
| Isopropyl Isostearate | 1.33 | 1.33 | 1.33 | 1.33 | 1.33 | 1.33 |
| Polymethylsilsesquioxane | 0.25 | 0.25 | 0.25 | 0.25 | 0.25 | 0.25 |
| Cetearyl Glucoside, | 0.20 | 0.20 | 0.20 | 0.20 | 0.20 | 0.20 |
| Cetearyl Alcohol | | | | | | |
| Behenyl Alcohol | 0.40 | 0.40 | 0.40 | 0.40 | 0.40 | 0.40 |
| Ethylparaben | 0.20 | 0.20 | 0.20 | 0.20 | 0.20 | 0.20 |
| Propylparaben | 0.10 | 0.10 | 0.10 | 0.10 | 0.10 | 0.10 |
| Cetyl Alcohol | 0.32 | 0.32 | 0.32 | 0.32 | 0.32 | 0.32 |
| Stearyl Alcohol | 0.48 | 0.48 | 0.48 | 0.48 | 0.48 | 0.48 |
| PEG-100 Stearate | 0.10 | 0.10 | 0.10 | 0.10 | 0.10 | 0.10 |
| Thickener: | | | | | | |
| Sepigel 305¹ | 2.00 | 2.00 | 2.00 | 2.00 | 2.00 | 2.00 |
| Oil Phase Part 2: | | | | | | |
| Benzyl Alcohol | 0.25 | 0.25 | 0.25 | 0.25 | 0.25 | 0.25 |
| DC 1503² | 2.00 | 2.00 | 2.00 | 2.00 | 2.00 | 2.00 |
| Active: | | | | | | |
| OCALINE³ | .01% | 12% | 0.1% | 6% | 2% | 5% |
| Total: | 100% | 100% | 100% | 100% | 100% | 100% |

¹Polyacrylamide, C13-14 isoparaffin, and laureth-7, from Seppic, France.
²Dimethicone and Dimethiconol from Dow Corning, Inc., Midland, MI.
³Available from Soliance US, Allendale, New Jersey

### Example 1—In Vivo Study—Treating Type 2 and Type 3 Periorbital Dyschromia

This example demonstrates the ability of pumpkin seed extract containing compositions of the present array to improve the appearance of Type II and Type III periorbital dyschromia. Twenty-five Caucasian female test subjects aged 20 to 60 were enrolled in a nine-week, split-face, round-robin design study to evaluate the ability of a pumpkin seed extract to improve the appearance of Type II and Type III periorbital dyschromia. The oil-in-water emulsion of Example R from Table 4 was evaluated in this study.

During the study, the under eye portion of the periorbital region (i.e., the shaded area 400 in FIG. 4A) on the left side of the test subject's face was treated with a test composition, and the under eye portion of the periorbital region on the right side of the test subject's face was treated with a vehicle control (i.e., the same composition as the test composition except without pumpkin seed extract). The test subjects were instructed to use cleansing cloths and a facial moisturizer, which were provided to them, twice a day. The test subjects were also instructed to refrain from using any eye treatment products during the course of the study and to avoid excessive UV exposure that could result in facial sunburn or tanning The test subjects were permitted to use their normal makeup products (e.g., foundation, blush, eye and lip liners) five minutes after application of the under eye compositions, but were asked not to switch brands. The test subjects applied the control and test compositions twice a day; once in the morning and once in the evening at least 30 minutes before going to bed. Approximately 0.04 g or 40-50 μl of each composition was applied to the periorbital skin under the appropriate eye. Images of the test subjects and blood perfusion data were collected at weeks 0 (baseline), 2, 4 and 8 for use in the

Imaging, Visual Perception Scale, and Blood Perfusion Methods described above. The baseline values were determined at the start of the test (week 0).

The results of the in vivo study are illustrated below in Tables 5A, 5B and 5C for test subjects exhibiting Type II periorbital dyschromia and in Tables 6A, 6B and 6C for test subjects exhibiting Type III periorbital dyschromia. The results illustrated in Tables 5A, 5B, 5C, 6A, 6B and 6C are averages of mean values, and the p-values are one-sided p-values generated using the ANOVA statistical method. Due to the small base size of the study, one-sided p-values of 0.2 or less are considered significant and one-sided p-values of greater than 0.2 but less than 0.3 are considered trending.

From the results it can be seen that the pumpkin seed extract provided an improvement in the appearance of both Type II and Type III periorbital dyschromia.

Table 5A below summarizes the results, using the Imaging method, of the use of 5% by weight of pumpkin seed extract compared with a control (chassis with no extract) at weeks 0, 2 and 4 when treating Type 2 periorbital dyschromia. Beneath Table 5A, those results are shown graphically for each of ΔL (higher is improved condition), Δa (lower is improved condition) and Δb (higher is improved condition).

Table 5B below summarizes the blood perfusion results, using the Laser Speckle method, of the use of 5% by weight of pumpkin seed extract compared with a control (chassis with no extract) at weeks 0, 2 and 4 when treating Type 2 periorbital dyschromia. Beneath Table 5B, those results are shown graphically.

Table 5C below summarizes the results, using the Visual Perception Scale, of the use of 5% by weight of pumpkin seed extract compared with a control (chassis with no extract) at weeks 0, 2 and 4 when treating Type 2 periorbital dyschromia.

TABLE 5C
Average VPS Type 2 Periorbital Dyschromia

| | Control | 5% | | | |
|---|---|---|---|---|---|
| | Chassis | Ocaline | | | |
| | (Change | (Change | Treatment- | Treatment- | |
| Endpoint/ | from | from | Control | Control | Direction |
| Timepoint | Baseline) | Baseline) | Chassis | (P = value*) | of better |
| Week 2 | −0.11 | 0.79 | 0.9 | [p = 0.036] | Higher |
| Week 4 | 0.39 | 1.03 | 0.64 | [p = 0.075] | Higher |

Table 6A below summarizes the results, using the Imaging method, of the use of 5% by weight of pumpkin seed extract compared with a control (chassis with no extract) at weeks 0, 2 and 4 when treating Type 3 periorbital dyschromia. Below Table 6A, those results are shown graphically for each of ΔL (higher is improved condition), Δa (lower is improved condition) and Δb (higher is improved condition).

Table 6B below summarizes the blood perfusion results, using the Laser Speckle method, of the use of 5% by weight of pumpkin seed extract compared with a control (chassis with no extract) at weeks 0, 2 and 4 when treating Type 3 periorbital dyschromia. Beneath Table 6B, those results are shown graphically.

Table 6C below summarizes the results, using the Visual Perception Scale, of the use of 5% by weight of pumpkin seed extract compared with a control (chassis with no extract) at weeks 2 and 4 when treating Type 3 periorbital dyschromia.

TABLE 6C
Average VPS Type 3 Periorbital Dyschromia

| | Control | 5% | | | |
|---|---|---|---|---|---|
| | Chassis | Ocaline | | | |
| | (Change | (Change | Treatment- | Treatment- | |
| Endpoint/ | from | from | Control | Control | Direction |
| Timepoint | Baseline) | Baseline) | Chassis | [P = value*] | of better |
| Week 2 | 0.63 | −0.29 | −0.92 | [p = 0.94] | Higher |
| Week 4 | −0.66 | −0.18 | 0.48 | [p = 0.19] | Higher |

### Example 2—In Vitro Study (B16—Melanin Inhibition Assay)

This example demonstrates the inability of pumpkin seed extract to inhibit melanin synthesis. It is believed that an overabundance of melanin is a key contributor to the appearance of Type I periorbital dyschromia, but not for Type II periorbital dyschromia. Thus, treating Type I periorbital dyschromia with pumpkin seed extract should not provide any improvement in its appearance, as demonstrated by the lack of melanin inhibition activity in a conventional B16 assay. This is important because it shows that a “one size fits all” approach may not be the best way to treat all types of periorbital dyschromia. For example, a composition that utilizes pumpkin seed extract may not improve the appearance of Type I periorbital dyschromia.

In this example, a commercially available B16-F1 mouse melanoma cell line from American Tissue Culture Collection, Virginia, USA was employed in a conventional melanin synthesis inhibition assay. The cell culture medium used in the assay is 500 mL of Dulbecco's Modified Eagle's Medium (DMEM), 50 mL Fetal Bovine Serum (FBS), and 5 mL of penicillin-streptomycin liquid. B16-F1 cells that are cultured in this medium and grown to greater than 90% confluency will synthesize melanin. While not intending to be bound by any theory, it is hypothesized that the melanin synthesis is stimulated by the culture medium and/or stress induced by growth to a high confluency. The DMEM and FBS can be obtained from American Tissue Culture Collection and the penicillin-streptomycin liquid can be obtained from Invitrogen, Inc., California, USA. Equipment used in the assay include a CO₂ incubator, such as a Forma Series Model 3110 by Therma Scientific, Massachusetts, USA; a hemocytometer, such as a Bright Line model by Hauser Scientific, Pennsylvania, USA; and a UV-Visible Spectrum Plate Reader, such as a SpectraMax250 from Molecular Devices, California, USA.

Day 0: To begin the assay, the cell culture medium is heated to 37° C. and 29 mL of the medium is placed into a T-150 flask. Approximately 1×10⁶ of B16-F1 passage 1 mouse cells are added to the T-150 flask and incubated for 3 days at 37° C., 5% CO₂, 90% relative humidity, until ˜80% confluency.

Day 3: The cells from the T-150 flask are trypsinized, and the concentration of cells is determined using the hemacytometer. Initiate a 96 well plate with 2,500 cells per well in 100 μL of cell culture medium. Incubate the plate at 37° C., 5% CO2, 90% relative humidity for 2 days until at least 20% to 40% confluent.

Day 5: Remove the cell culture medium from the plate and replace with fresh culture medium (100 uL per well). Add 1 uL of test compound diluted in a water solvent. Multiple dilution ratios may be tested in order to generate a dose response curve, wherein preferably three wells are treated with each dilution ratio. Positive and negative controls may include wells having the cell culture medium, B16-F1 cells, and the solvent (negative control), and wells comprising the cell culture medium, B16-F1 cells and a known melanin inhibitor (e.g., deoxyarbutin or kojic acid).

Day 7: Cells should have a confluency greater than ˜90%. If not, this data point is not used. Add 100 uL of a 0.75% sodium hydroxide solution to each well. Read the 96 well plate using the UV-Vis Plate Reader at 410 nm to optically measure the amount of melanin produced between wells that are treated with the pumpkin seed extract and control wells that are not. Wells in which melanin is produced appear brownish in color. Wells in which little melanin is produced appear clear to light purple in color. Percentage of melanin synthesis inhibition is calculated by the following equation:

$$\frac{100 - [ OD 410 Test Compound - OD 410 Control \#2 ] \times 100}{( OD 410 Control \#1 - OD 410 Control \#2 )}$$

Where OD410 is the Optical Density at 410 nm as measured by the UV-Vis Spectrum Plate Reader.

When Control #3 is used, the formula for percentage melanin synthesis inhibition is:

$$\frac{100 - [ OD 410 Test Compound - OD 410 Control \#3 ] \times 100}{( OD 410 Control \#1 - OD 410 Control \#2 )}$$

The concentration of test agent needed to provide the IC 50 is recorded.

Table 7 shows the concentration of each composition needed to provide the IC 50. The positive controls used in this example are deoxyarbutin and kojic acid, both of which are well-known inhibitors of melanin synthesis. As shown in Table 7, the concentration of the test composition required to obtain IC 50 was much higher than either the positive control or the kojic acid, indicating that the pumpkin seed extract tested in this example is a poor inhibitor of melanin synthesis.

TABLE 7
B16 (IC 50)

| | Concentration |
|---|---|
| | Needed for IC |
| Composition | 50 (v/v) |
| Deoxyarbutin | 0.008 |
| Kojic Acid | 0.01 |
| OCALINE¹ | 0.4 |

¹Pumpkin seed extract available from Soliance (a subsidiary of Givaudan), Allendale, NJ

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Additionally, properties described herein may include one or more ranges of values. It is to be understood that these ranges include every value within the range, even though the individual values in the range may not be expressly disclosed.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES/COMBINATIONS

A. An array of products for treating periorbital dyschromia, comprising:

a. a first product that improves the appearance of a first type of periorbital dyschromia but not a second type of periorbital dyschromia; and

b. a second product that improves the appearance of a second type of periorbital dyschromia but not the first type of periorbital dyschromia, wherein the second product comprises an effective amount of an extract of Cucurbita pepo.

B. The array of paragraph A, wherein the first product provides an increase in L value in Type I periorbital dyschromia.
C. The array of paragraph A or B, wherein the first product comprises an agent selected from the group consisting of haloxyl, niacinamide, undecylenoyl phenylalanine, salicylic acid, chenopodium Quinoa Seed Extract, propyl gallate, gallyl glucoside, epigallocatechin gallatyl glucoside, and mixtures thereof
D. The array of any one of paragraphs A-C, wherein the first product comprises two compositions used in combination, the first composition comprising haloxyl and the second composition comprising an agent selected from the group consisting of niacinamide, undecanoyl phenylalanine, salicylic acid, and mixtures thereof.
E. The array of any of paragraphs A-D, wherein the second product provides increased lightness.
F. The array of any one of paragraphs A-E, further comprising a third product that improves the appearance of a third type of periorbital dyschromia, but does not improve the appearance of at least one of the first and second types of periorbital dyschromia.
G. The array of paragraph F, wherein the third product includes a cosmetic agent selected from the group consisting of an effective amount of an extract of Cucurbita pepo, palmitoyl tetrapeptide-7, propyl gallate, gallyl glucoside, epigallocatechin gallatyl glucoside, palmitoyl oligopeptide, palmitoyl tetrapeptide 7, and mixtures thereof.
H. The array of any one of paragraphs A-G, wherein the first and second products each include a package and each package includes common indicia and product-specific indicia.
I. The array of any one of paragraphs A-H, wherein the second product provides an improvement in Visual Perception Scale in Type II periorbital dyschromia.
J. The array of paragraph F or G, wherein the third product provides a decrease in a* value in Type III periorbital dyschromia.
K. The array of any one of paragraphs F, G or J, wherein the third product provides an increase in L* value in Type III periorbital dyschromia.
L. The array of any one of paragraphs F, G, J or K, wherein the third product provides an improvement in Visual Perception Scale in Type III periorbital dyschromia.
M. The array of any one of paragraphs A-L, wherein the first product reduces melanin production as measured in the BC 16 melanin inhibition assay.
N. The array of any one of paragraphs A-M, wherein the first product is a component of a collection of products for periorbital skin benefits.
O. An array of products for treating periorbital dyschromia, comprising:
a. a first product that improves the appearance of Type I periorbital dyschromia, the first product comprising a first and a second composition to be used in combination, the first composition comprising haloxyl and the second composition comprising an agent selected from the group consisting of niacinamide, undecanoyl phenylalanine, salicylic acid, chenopodium Quinoa Seed Extract, propyl gallate, gallyl glucoside, epigallocatechin gallatyl glucoside and mixtures thereof;
b. a second product that improves the appearance of Type II periorbital dyschromia, wherein the second product comprises an effective amount of an extract of Cucurbita pepo; and
c. a third product that includes a cosmetic agent selected from the group consisting of an effective amount of an extract of Cucurbita pepo, palmitoyl tetrapeptide-7, propyl gallate, gallyl glucoside, epigallocatechin gallatyl glucoside, palmitoyl oligopeptide, palmitoyl tetrapeptide 7, and mixtures thereof.

1. An array of products for treating periorbital dyschromia, comprising:
a. a first product that improves the appearance of a first type of periorbital dyschromia but not a second type of periorbital dyschromia; and
b. a second product that improves the appearance of a second type of periorbital dyschromia but not the first type of periorbital dyschromia, wherein the second product comprises an effective amount of an extract of Cucurbita pepo.

2. The array of claim 1, wherein the first product provides an increase in L value in Type I periorbital dyschromia.

3. The array of claim 1, wherein the first product comprises an agent selected from the group consisting of niacinamide, undecylenoyl phenylalanine, salicylic acid, chenopodium Quinoa Seed Extract, propyl gallate, gallyl glucoside, epigallocatechin gallatyl glucoside, and mixtures thereof

4. The array of claim 3, wherein the first product comprises two compositions used in combination, the first composition comprising haloxyl and the second composition comprising an agent selected from the group consisting of niacinamide, undecanoyl phenylalanine, salicylic acid, and mixtures thereof.

5. The array of claim 1, wherein the second product provides increased lightness.

6. The array of claim 1, further comprising a third product that improves the appearance of a third type of periorbital dyschromia, but does not improve the appearance of at least one of the first and second types of periorbital dyschromia.

7. The array of claim 6, wherein the third product includes a cosmetic agent selected from the group consisting of an effective amount of an extract of Cucurbita pepo, palmitoyl tetrapeptide-7, propyl gallate, gallyl glucoside, epigallocatechin gallatyl glucoside, palmitoyl oligopeptide, palmitoyl tetrapeptide 7, and mixtures thereof.

8. The array of claim 1, wherein the first and second products each include a package and each package includes common indicia and product-specific indicia.

9. The array of claim 8, wherein the common indicia is a brand name and product-specific indicia corresponds to a type of periorbital dyschromia.

10. The array of claim 8, wherein the product-specific indicia depicts an illustration of periorbital dyschromia disposed on an image of a face.

11. The array of claim 1, wherein the second product provides an improvement in Visual Perception Scale in Type II periorbital dyschromia.

12. The array of claim 6, wherein the third product provides a decrease in a* value in Type III periorbital dyschromia.

13. The array of claim 6, wherein the third product provides an increase in L* value in Type III periorbital dyschromia.

14. The array of claim 6, wherein the third product provides an improvement in Visual Perception Scale in Type III periorbital dyschromia.

15. The array of claim 1, wherein the first product reduces melanin production as measured in the BC 16 melanin inhibition assay.

16. The array of claim 1, wherein the first product is a component of a collection of products for periorbital skin benefits.

17. An array of products for treating periorbital dyschromia, comprising:
a. a first product that improves the appearance of Type I periorbital dyschromia; and
b. a second product that improves the appearance of Type II periorbital dyschromia, Type III periorbital dyschromia, or both Type II and Type III periorbital dyschromia, wherein the second product does not improve the appearance of Type I periorbital dyschromia and wherein the second product comprises an effective amount of an extract of Cucurbita pepo.

18. The array of claim 17 further comprising a third product that improves the appearance of Type III periorbital dyschromia.

19. The array of claim 18 wherein the third product includes a cosmetic agent selected from the group consisting of an effective amount of an extract of Cucurbita pepo, palmitoyl tetrapeptide-7, propyl gallate, gallyl glucoside, epigallocatechin gallatyl glucoside, palmitoyl oligopeptide, palmitoyl tetrapeptide 7, and mixtures thereof.

20. An array of products for treating periorbital dyschromia, comprising:
a. a first product that improves the appearance of Type I periorbital dyschromia, the first product comprising a first and a second composition to be used in combination, the first composition comprising haloxyl and the second composition comprising an agent selected from the group consisting of niacinamide, undecanoyl phenylalanine, salicylic acid, chenopodium Quinoa Seed Extract, propyl gallate, gallyl glucoside, epigallocatechin gallatyl glucoside and mixtures thereof;
b. a second product that improves the appearance of Type II periorbital dyschromia, wherein the second product comprises an effective amount of an extract of Cucurbita pepo; and
c. a third product that includes a cosmetic agent selected from the group consisting of an effective amount of an extract of Cucurbita pepo, palmitoyl tetrapeptide-7, propyl gallate, gallyl glucoside, epigallocatechin gallatyl glucoside, palmitoyl oligopeptide, palmitoyl tetrapeptide 7, and mixtures thereof.
